SEQUENCE LISTING (3 sequences)

SEQ ID NO: 1 - RNA, 20 nt, Synthetic construct
  source 1..20: mol_type = other RNA; note = Nucleic acid
atattctgttttcagagcag

SEQ ID NO: 2 - RNA, 20 nt, synthetic construct
  source 1..20: mol_type = other RNA; note = Nucleic acid
ttccagtacctctgggaagc

SEQ ID NO: 3 - RNA, 20 nt, synthetic construct
  source 1..20: mol_type = other RNA; note = Nucleic acid
gaagataaacggaaaacaac

(19) United States
(12) Patent Application Publication
(10) Pub. No.: US 2026/0297527 A1
(43) Pub. Date: Oct. 1, 2026

(54) Hypoxia-Resistant Immune Cells for Sustained Persistence and Functionality in the Hypoxic Tumor Microenvironment

(71) Applicant: UNIVERSITEIT ANTWERPEN, Antwerpen (BE)

(72) Inventors: Jorrit De Waele, Wilrijk (BE); Evelien Smits, Wilrijk (BE); Tias Verhezen, Wilrijk (BE); An Wouters, Wilrijk (BE)

(21) Appl. No.: 19/479,002

(22) PCT Filed: Apr. 26, 2024
(86) PCT No.: PCT/EP2024/061596

§ 371 Date: Oct. 27, 2025

(30) Foreign Application Priority Data
Apr. 28, 2023 (EP) 23170718.3
Oct. 27, 2023 (EP) 23206436.0

(51) Int. Cl. C12N 5/0783; A61K 35/17; A61P 35/00; C12N 9/14

(52) U.S. Cl. CPC C12N 5/0646; A61K 35/17; A61P 35/00; C12N 9/14; C12N 2510/00; C12Y 306/05005

(57) ABSTRACT

The invention concerns a population of hypoxia-resistant immune cells, such as hypoxia-resistant T cells or Natural Killer (NK) cells for use as a medicament. The hypoxia-resistant immune cells are considered for use in treating different cancers such as haematological malignancies and solid tumours. The invention further concerns methods for producing said hypoxia-resistant immune cells and pharmaceutical compositions comprising said hypoxia-resistant immune cells.

### CROSS REFERENCE TO RELATED APPLICATIONS

This application is a national phase entry under 35 U.S.C. § 371 of International Patent Application PCT/EP2024/061596, filed Apr. 26, 2024, designating the United States of America and published in English as International Patent Publication WO2024/223860 on Oct. 31, 2024, which claims the benefit under Article 8 of the Patent Cooperation Treaty to European Patent Application Serial No. 23170718.3, filed Apr. 28, 2023, and European Patent Application Serial No. 23206436.0, filed Oct. 27, 2023, the entireties of which are hereby incorporated by reference.

### FIELD OF THE INVENTION

The present invention relates broadly to the field of cell therapy. Particularly, the invention relates to hypoxia-resistant immune cells that have reduced or inhibited Drp1 expression and/or activity for use in treating different cancer and tumour types, and pharmaceutical compositions comprising said cells. The invention further concerns methods of producing immune cells that have improved hypoxia resistance.

### BACKGROUND OF THE INVENTION

Cancer remains a major global health issue, being the second leading cause of premature death worldwide. The burden of cancer incidence and mortality keeps growing, causing immense physical, emotional and financial strain on individuals, families and health systems. While it has been established that distinct malignancies display markedly different characteristics, certain common features of particular tumour types have been described. For solid tumours in particular, it is well established that said tumours generally contain regions characterised by decreased oxygen levels, i.e. hypoxic regions. The degree of hypoxia within such a solid tumour is highly heterogenous (ranging from mild hypoxic to essentially anoxic levels) and is subject to changes as the tumour enlarges. Hypoxic regions in solid tumours are known to impact numerous cellular phenomena within the tumour including cellular responses and interactions between different cell types in the tumour microenvironment, for example interactions between immune cells and tumour cells. Hypoxic tumour microenvironments are generally considered to be associated with tumour progression, increased tumour aggressiveness, higher metastasis probability, and increased tumour resistance to radio- and chemotherapy.

Cancer immunotherapy is becoming available as a potent tool for medical practitioners to treat cancer. Generally, these immune-targeting therapies are characterised by a lower burden for the patient when compared to traditional anticancer agents such as chemotherapy. One approach of particular interest is cell therapy (such as Chimeric Antigen Receptor (CAR) T-cell therapy) where engineered immune cells are modified (i.e. engineered) outside of the body of the patient to improve their anti-tumour potential prior to their reintroduction into patient. It is believed that the adoption of such cell therapies will continue to increase with improvements in engineering strategies, scaling solutions for production, and improved regulatory frameworks.

To date, most strategies to reinvigorate NK cells for cancer therapy have been focusing on blocking inhibitory cell receptors, CAR engineering or cytokine stimulation. Recent studies have observed that the proper functioning of immune cells to effectively act against (solid) tumours is greatly hampered by the hypoxic tumour microenvironment, resulting to mitochondrial fragmentation (i.e. mitochondrial fission) of said immune cells (Zheng et al., Mitochondrial fragmentation limits NK cell-based tumor immunosurveillance, Nat Immunol, 2019). Fragmentation of mitochondria drives Reactive Oxygen Species (ROS) production, apoptosis, and mitophagy. Thus, hypoxia is detrimental for the potency of the immune cells of the subject to clear the body of tumour cells, and may be considered an effective tumour-mediated immune evasion route.

There is therefore an ongoing need for therapies and treatment strategies that are resistant to and/or even exploit the harsh cellular conditions of tumours, particularly hypoxic microenvironments, which can be translated into an off-the-shelf cell product with a superior safety profile and further push the boundaries of cancer treatment. Ultimately, this would lead to improved survival rates and a reduced societal and individual burden of cancer.

### SUMMARY OF THE INVENTION

By means of thorough experimentation, the inventors have generated immune cells that are particularly suitable for use in hypoxic tumour conditions. Unexpectedly, the immune cells generated by the methods described herein display improved properties over hypoxia-resistant immune cells described in the art, such as an improved potency to survive hypoxic conditions. More particularly, mitochondrial fragmentation is inhibited in said cells by the treatments described further herein. Without wishing to be bound by theory, it is hypothesized that the improved survival rates of the generated immune cells bring forth tangible advantages when used as therapeutic agents since the cells will be active over a prolonged period upon introduction into a hypoxic tumour environment. The finding is of particular importance because of its broad applicability to different types of immune cells (e.g. Natural Killer (NK) cells, T cells) and sources thereof (primary cells, cultured cells), effectively aiding in providing modified immune cells as an off-the-shelf cellular cancer treatment, which is of particular interest in the field of redirected immune cells (e.g., CAR cells, or cells equipped with bispecific or even multispecific affinity ligands). Moreover, surprisingly the inventors have found that Drp1 knock-out cells that are further optionally endowed with further favourable properties are viable, which is unexpected given the central role of Drp1 as mitochondrial fission-regulating GTPase, and studies that report on the essential character of the protein for proper embryonic development, and a loss of Drp1 results in aberrant brain development and early mortality.

Accordingly, a first aspect of the invention provides a population of immune cells wherein Drp1 expression and/or activity is directly (and thus specifically) reduced or inhibited (i.e., resulting in “modified” immune cells), for use as a medicament. Hence, it is to be understood that the cells subject of the present invention have a decreased Drp1 expression and/or activity compared to a cell of the same type not comprising said modification of Drp1 expression and/or activity, and that preferably the extent of Drp1 decrease is at least 50%. More particularly, a first aspect of the invention provides a population of hypoxia-resistant immune cells wherein Drp1 expression and/or activity is directly (and thus specifically) reduced or inhibited, for use as a medicament. Preferably, each cell within said population of hypoxia-resistant immune cells is characterized by a reduced or inhibited Drp1 expression. Said hypoxia-resistant immune cells are characterized by improved resistance to hypoxic conditions when compared to their naturally occurring (i.e. wild-type), or unmodified, or non-engineered counterparts. Hence, the hypoxia resistance and the reduced or inhibited Drp1 expression may be observed when compared to respectively the hypoxia resistance of, and the reduced or inhibited Drp1 expression in, a non-hypoxia-resistant immune cell which otherwise shares a genomic, transcriptomic, and/or proteomic identity to the hypoxia-resistant immune cell. The findings described herein are particularly envisaged to further modify and augment properties of for example redirected immune cells such as CAR cells or cells further characterised by their expression of or interaction with monospecific affinity ligands, bispecific or even multispecific affinity ligands. The invention equally provides in pharmaceutical compositions comprising said population of (optionally redirected) hypoxia-resistant immune cells. The present invention thus also provides methods of treating a subject with a population of (optionally redirected) hypoxia-resistant immune cells wherein Drp1 expression and/or activity is directly reduced or inhibited, or with a pharmaceutical composition comprising said population of hypoxia-resistant immune cells.

Preferably, the population of (optionally redirected) hypoxia-resistant immune cells or pharmaceutical composition is for use in treating cancer, more preferably a cancer that is characterised by, or associated with the presence of a hypoxic tumour microenvironment. Hence, the invention additionally provides in a method for treating cancer, preferably a cancer that is characterised by, or associated with the presence of a hypoxic tumour microenvironment, comprising administering to a subject in need thereof a (optionally redirected) hypoxia-resistant immune cell wherein Drp1 expression and/or activity is directly reduced or inhibited, or a pharmaceutical composition comprising said cells. Further envisaged is the use of a (optionally redirected) hypoxia-resistant immune cell wherein Drp1 expression and/or activity is directly reduced or inhibited for the manufacture of a medicament for the treatment of cancer, preferably a cancer that is characterised by, or associated with the presence of a hypoxic tumour microenvironment. Also envisaged is the use of a (optionally redirected) hypoxia-resistant immune cell wherein Drp1 expression and/or activity is directly reduced or inhibited, or a pharmaceutical composition comprising said cells, for the treatment of cancer, preferably a cancer that is characterised by, or associated with the presence of a hypoxic tumour microenvironment.

In particular embodiments, the population of (optionally redirected) hypoxia-resistant immune cells or pharmaceutical composition is for use in treating leukaemia (or any haematological malignancy) in a subject wherein the subject having leukaemia (or any haematological malignancy) is further characterized by the presence of a hypoxic bone marrow microenvironment.

In particular embodiments, the population of (optionally redirected) hypoxia-resistant immune cells or pharmaceutical composition is for use in treating a solid tumour in a subject. Optionally, the solid tumour is a solid tumour selected from the group consisting of: liver tumours, pancreatic tumours, colorectal tumours, breast tumours, head tumours, and neck tumours, and lung tumours.

In particular embodiments, the population of (optionally redirected) hypoxia-resistant immune cells or pharmaceutical composition is for use in allogenic cell therapy.

In particular embodiments, Drp1 expression and/or activity is directly reduced or inhibited by contacting said cells with an inhibitor that specifically targets Drp1, preferably wherein said inhibitor is a chemical inhibitor, a binding protein, a gene editing system, or an antisense agent.

In further embodiments of any of the populations or methods described herein, the Drp1 inhibitor is a small molecule inhibitor, preferably the Drp1 inhibitor is Mitochondrial division inhibitor 1 (Mdivi-1).

Optionally, in the population and methods described herein, Drp1 expression and/or activity of the immune cells is directly reduced or inhibited by genomic modification of the DNM1L gene or DNM1L promoter sequence. In further embodiments, the genetic modification is a deletion, substitution, and/or insertion of one or more nucleotides in the DNM1L gene or DNM1L promoter sequence.

In particular embodiments, the (optionally redirected) hypoxia-resistant immune cells that are optionally comprised in a pharmaceutical composition are Drp1 knock-out cells. Optionally, the immune cells are conditional Drp1 knock-out cells.

In preferred embodiments, the immune cells are human immune cells. In yet further preferred embodiments, the subject to be treated with said immune cells is a human subject.

In particular embodiments of the methods and populations described herein, the immune cells are immune cells derived from peripheral blood.

Optionally, the immune cells are natural killer cells or T cells. In further embodiments, the natural killer cells are primary natural killer cells or immortalized natural killer cells. Optionally, the immortal natural killer cells are NK-92 cells or NK-92 MI cells.

Optionally, in addition to having a reduced or inhibited Drp1 expression level and/or activity, the immune cells envisaged in the present invention are further modified (i.e. “redirected”) to express a chimeric antigen receptor (CAR) and/or a heterologous T cell receptor or artificial T cell receptor. Optionally, the CAR is a CAR that specifically binds to a tumoral antigen. Optionally, the CAR is 70-CAR-IL-15. Alternatively or in combination with the above, in addition to having a reduced or inhibited Drp1 expression level and/or activity, the immune cells envisaged in the present invention are further modified (i.e. “redirected”) to express, display, secrete and/or interact with a mono- or multispecific affinity ligand such as but not limited to a bispecific antibody. Optionally, the multispecific affinity ligand binds at least both to the surface of the redirected immune cell and to an antigen, by means of example and not limitation a tumoral antigen.

Optionally, in addition to having a reduced or inhibited Drp1 expression level and/or activity and possible further expressing a CAR, the immune cells are further modified for survival and/or efficacy in a solid tumour microenvironment.

In certain embodiments, the solid tumour envisaged to be treated is a solid tumour having local oxygen levels of less than about 8%, preferably of less than about 5%, more preferably of less than about 3%, most preferably of less than about 1%.

In certain embodiments, the Drp1 expression and/or activity in the population of hypoxia-resistant immune cells which may be comprised in a pharmaceutical composition, is reduced for a period of at least about 72 hours prior to introduction into (i.e. administration to) a subject.

Optionally, the immune cells comprise a kill switch mechanism. Alternatively or in addition, the immune cells can be auxotrophic immune cells.

In certain embodiments of the populations, pharmaceutical compositions and methods envisaged herein, the Drp1 expression and/or activity is reduced in the population of hypoxia-resistant immune cells by at least about 50%, preferably at least about 75%, preferably at least about 85%, more preferably at least about 95%, most preferably by about 100% when compared to a non-hypoxia-resistant immune cell.

Optionally, the pharmaceutical composition comprises at least one (i.e. one or more) excipients.

In a related aspect, the invention concerns a method of producing an (optionally redirected) hypoxia-resistant immune cell or a population of hypoxia-resistant immune cells, such as those described above, having reduced or inhibited (i.e., decreased) Drp1 expression and/or activity when compared to a cell type not comprising said modification of Drp1 expression and/or activity comprising the steps of:
providing (optionally redirected) immune cells;
treating said immune cells ex vivo with a specific Drp1 inhibitor. In embodiments wherein redirected immune cells are envisaged it is evident that the immune cells may be treated Drp1 inhibitor prior to, concomitant with, or after one or more manipulation steps to redirect said immune cells.

In preferred embodiments, the specific Drp1 inhibitor is an inhibitor selected from the group consisting of: a chemical inhibitor, a binding protein, a gene editing system, or an antisense agent. In further embodiments, the specific Drp1 inhibitor is a chemical inhibitor that is contacted with the immune cells for a period of about 1 to about 100 hours, preferably for a period of about 48 to about 72 hours, more preferably for a period of about 72 hours. In yet further embodiments, the chemical inhibitor is Mitochondrial division inhibitor 1 (Mdivi-1). Optionally, the immune cells are treated with from about 1 to about 100 μM of Mdivi-1, preferably from about 5 μM to about 25 μM of Mdivi-1, more preferably with about 10 μM of Mdivi-1.

Preferably in the methods envisaged hereinabove, the Drp1 expression and/or activity is reduced in the (population of) immune cells by at least about 75%, preferably at least about 85%, more preferably at least about 95%, most preferably by about 100% by the specific inhibitor.

Optionally, the specific Drp1 inhibitor is a gene editing system, preferably a targeted nuclease gene editing system, more preferably a CRISPR/Cas gene editing system, most preferably a CRISPR/Cas9 gene editing system.

In certain embodiments of the method, the cells are treated with a further specific inhibitor for reducing expression of a further protein.

In further embodiments of the method of production envisaged herein, the method comprises a further step of introducing one or more heterologous genes into the genomic sequence of the immune cells. A preferred heterologous gene in the present context is a chimeric antigen receptor. Optionally, the chimeric antigen receptor is CD70-CAR-IL-15. Alternatively or in combination with the above, the method comprises a step of further modifying (i.e. “redirecting”) the immune cells to express, display, secrete and/or interact with a mono- or multispecific affinity ligand such as but not limited to a bispecific antibody. Optionally, the bispecific affinity ligand binds both to the surface of the redirected immune cell and to an antigen, by means of example and not limitation a tumoral antigen.

Optionally, the immune cells are natural killer (NK) cells, such as but not limited to redirected natural killer cells.

The invention is also directed to a population of hypoxia-resistant immune cells, obtained by any embodiment of the methods described herein. Redirected hypoxia-resistant immune cells obtained by any embodiment of the methods described herein are also envisaged Optionally, the hypoxia-resistant immune cells are CD70-CAR-IL-15 NK-92 cells.

A further related aspect of the invention provides a kit of parts that comprises means to generate a population of (optionally redirected) hypoxia-resistant immune cells wherein Drp1 expression and/or activity is directly reduced or inhibited, and wherein the kit of parts further comprises instructions for use.

The above and further aspects and preferred embodiments of the invention are described in the following sections and in the appended claims. The subject matter of the appended claims is hereby specifically incorporated in this specification.

### BRIEF DESCRIPTION OF THE DRAWINGS

FIG. 1. Viability of control, Mdivi-1 treated and Drp1^{KO} NK-92 cells after 48 h culturing in 1% and 21% O₂. The Graphs show the percentage viable NK-92 cells. The percentage viable NK-92 cells are identified as Annexin V− and/or 7-AAD− cells with flow cytometry. Drp1^{KO} NK-92 cells have a significantly better viability after hypoxic culturing compared to control NK-92 cells. Error bars represent standard deviation. * P≤0.05, *** P≤0.001. KO: knock out. Samples from left to right: 21% O₂ control; 1% O₂ control; 1% O₂ Mdivi-1 treated; 1% O₂ Drp1^{KO}.

FIG. 2. In vitro cytotoxic potential of control, Mdivi-1 treated and Drp1^{KO} NK-92 cells after 48 h culturing in 1% and 21% O₂. The graphs show the percentage cell death of K562 target cells after 4 and 16 hours of co-culture with the different NK-92 cell conditions. The percentage dead target cells are identified as Annexin V+ and/or 7-AAD+ cells with flow cytometry. Mdivi-1 treated and Drp1^{KO} NK-92 cells have a significantly better killing capacity after hypoxic culturing compared to control NK-92 cells. Error bars represent standard deviation. *** P≤0.001. KO: knock out. Samples from left to right: 21% O₂ control; 1% O₂ control; 1% O₂ Mdivi-1 treated; 1% 02 Drp1^{KO}. An equal number of cells were used for each condition.

FIGS. 3A and 3B. In vitro cytotoxic potential of wild type (WT) and Drp1^{KO} NK-92 cells after 48 h culturing in 1% and 21% O₂. The ratio cancer cells to NK-92 cells is 1 to 5. FIG. 3A) normalized cell index determined by xCELLigence of Panc-1 target cells during 24 hours of co-culture with the different NK-92 cell conditions. FIG. 3B) Percentage dead target cells identified by the ratio of Panc-1 cell index alone and the cell index of the conditions of interest at endpoint (24 h). Error bars represent standard deviation of two technical replicates.

FIGS. 4A and 4B. In vitro cytotoxic potential of wild type (WT) and Drp1^{KO} NK-92 cells after 48 h culturing in 1% and 21% O₂. The ratio cancer cells to NK-92 cells is 1 to 1. FIG. 4A) normalized cell index determined by xCELLigence of Panc-1 target cells during 24 hours of co-culture with the different NK-92 cell conditions. FIG. 4B) Percentage dead target cells identified by the ratio of Panc-1 cell index alone and the cell index of the conditions of interest at endpoint (24 h). Error bars represent standard deviation of two technical replicates.

FIGS. 5A-5C. In vitro cytotoxic potential against several solid tumour cell lines of wild type (WT) and Drp1K° NK-92 cells after 48 h culturing in 1% and 21% O₂. The ratio cancer cells to NK-92 cells is 1 to 5. Normalized cell index determined by xCELLigence of solid tumour cells (FIG. 5A: HeLa, FIG. 5B: LIM2099, FIG. 5C: SC263) during 24 hours of co-culture with the different NK-92 cell conditions. Error bars represent standard deviation of two technical replicates. KO(1) and KO(2) represent two individual biological DRP1^{KO} CAR NK cell replicates.

FIGS. 6A-6C. In vitro cytotoxic potential of wild type (WT) and Drp1^{KO} NK-92 cells after 48 h culturing in 1% and 21% O₂. The ratio cancer cells to NK-92 cells is 1 to 1. Normalized cell index determined by xCELLigence of solid tumour cells (FIG. 6A: HeLa, FIG. 6B: LIM2099, FIG. 6C: SC263) during 24 hours of co-culture with the different NK-92 cell conditions. Error bars represent standard deviation of two technical replicates. KO(1) and KO(2) represent two individual biological DRP1^{KO} CAR NK cell replicates.

FIG. 7. Summary of the in vitro cytotoxic potential against solid tumour cells of wild type (WT) and Drp1^{KO} NK-92 cells after 48 h culturing in 1% and 21% O₂. The ratio cancer cells to NK-92 cells is 1 to 5. Percentage dead target cells identified by the ratio of solid tumour cell index alone and the cell index of the conditions of interest at endpoint (24 h) during the xCELLigence coculture assay. Dots represent biological replicates.

FIG. 8. In vitro cytotoxic potential of wild type (WT) and Drp1^{KO} NK-92 cells after 48 h culturing in 1% and 21% O₂. The ratio cancer cells to NK-92 cells is 1 to 5. The graph shows the percentage cell death of Raji target cells after 4 hours of co-culture with the different NK-92 cell conditions. The percentage dead target cells are identified as Annexin V+ and/or 7-AAD+ cells with flow cytometry. Each dot represents a biological replicate. Error bars represent standard deviation. Left bar of each condition: 21% O₂. Right bar of each condition: 1% O₂.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g. any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e. also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

Unless indicated otherwise, all methods, steps, techniques and manipulations that are not specifically described in detail can be performed and have been performed in a manner known per se, as will be clear to the skilled person. Reference is for example again made to standard handbooks as well as to the general background art referred to herein and to the further references cited therein.

Any genes of interest disclosed in the context of the findings made by the inventors are in addition to their full name indicated by their commonly accepted GeneCards Symbol (https://www.genecards.org/). A skilled person appreciates that described herein may equally be annotated by alternative identifiers such as but not limited to their HGNC (https://www.genenames.org/), NCBI Entrez Gene (https://www.ncbi.nlm.nih.gov/gene/), Ensembl (http://www.ensembl.org/), or UniProtKB identifier (https://www.uniprot.org). A skilled person readily appreciates that any sequences represented in sequence databases may be of precursors of markers, peptides, polypeptides, proteins, or nucleic acids and may include parts which are processed away from mature molecules.

It is appreciated that a skilled person is capable of assessing sequence identity between sequences. Since methods and tools to verify sequence identity between different sequences of amino acids or nucleic acids are well known. Such tools include (Protein) BLAST, ClustalW2, SIM alignment tool, TranslatorX, and T-COFFEE. The percentage of identity between two sequences may show minor differences depending on the algorithm choice and parameters. The term “sequence identity” as used herein refers to the relationship between sequences at the nucleotide (or amino acid) level. The expression “% identical” is determined by comparing optimally aligned sequences, e.g. two or more, over a comparison window wherein the portion of the sequence in the comparison window may comprise insertions and/or deletions as compared to the reference sequence for optimal alignment of the sequences. The reference sequence does not comprise insertions or deletions. A reference window is chosen and the “% identity” is then calculated by determining the number of nucleotides (or amino acids) that are identical between the sequences in the window, dividing the number of identical nucleotides (or amino acids) by the number of nucleotides (or amino acids) in the window and multiplying by 100. Unless indicated otherwise, the sequence identity is calculated over the whole length of the reference sequence. An example procedure to determine the percent identity between a particular amino acid sequence and the amino acid sequence of a query polypeptide will entail aligning the two amino acid sequences using the Blast 2 sequences (B12seq) algorithm, available as a web application or as a standalone executable programme (BLAST version 2.2.31+) at the NCBI web site (www.ncbi.nlm.nih.gov), using suitable algorithm parameters. An example of suitable algorithm parameters include: matrix=Blosum62, cost to open a gap=11, cost to extend a gap=1, expectation value=10.0, word size=3). A skilled person readily appreciates that any sequences represented in sequence databases or in the present specification may be of precursors of peptides, polypeptides, proteins, or nucleic acids and may include parts which are processed away from mature molecules.

Reference throughout the present description is made to terms such as “expression”, “expression level”, “quantity”, “amount”, “value”, and “level”, which each indicate a quantification of a gene expression level in a biological sample, in the context of the present disclosure the biological sample is typically an immune cell or a collection (i.e. population) of immune cells. Said quantitation may be an absolute or alternatively a relative quantification of a gene expression level in a biological sample. Relative quantification in the present context refers to the expression of a gene expression level relative to another value such as relative to a reference value, or even a reference range (e.g. a reference indicating a base-line expression of a marker in a given tissue), or relative to the expression level in a reference biological sample (i.e. indicated interchangeably herein by the term “baseline expression value”). These values or ranges can be obtained from a single biological sample or from a plurality of biological samples (i.e. biological repeats) and may be established by determining the Drp1 expression level in a single immune cell or a population of immune cells. Such a population of cells may comprise without limitation at least 2, preferably at least 10, more preferably at least 100, most preferably at least several hundred immune cells. The value may be obtained by a single measurement of the gene expression level in a biological sample, or by repeated measurement of the gene expression level in a biological sample (i.e. technical repeats).

An absolute quantitation of a gene expression level in a biological sample may be expressed by various measurement units such as but not limited to weight, molar amount, concentration (e.g. weight per volume or mol per volume), intensity, or copy number. A relative quantitation of a gene expression level in a biological sample may be expressed by various measurement units such as but not limited to increase, decrease, fold-increase, or fold-decrease relative to a reference gene expression level. The inventors have surprisingly found that immune cells having a reduced or inhibited Drp1 expression level and/or activity are viable over prolonged periods of time. Moreover, upon introduction into a hypoxic tumour microenvironment it was observed that the hypoxia-resistant immune cells showed an improved viability. Said hypoxia-resistant immune cells are characterized by improved resistance to hypoxic conditions when compared to their naturally occurring (i.e. wild-type), or unmodified, or non-engineered counterparts. This improved viability resulted in improved cytotoxicity of tumour cells in hypoxic tumour microenvironments. The marked improvements observed by the inventors in the manipulated immune cells remained present upon applying the manipulation in redirected immune cells. The redirected properties can be introduced either prior to, concomitant, or after the manipulation of the Drp1 expression level and/or activity.

Accordingly, the invention provides for the therapeutic use of a population immune cells which are characterized in that they are hypoxia-resistant as a result of a reduction or inhibition of Drp1 expression and/or activity in these cells. The application thus provides a population of hypoxia-resistant immune cells wherein Drp1 expression and/or activity is directly reduced or inhibited, for use as a medicament. The invention is thus also directed to the use of a population or group of hypoxia-resistant immune cells wherein Drp1 expression and/or activity is directly reduced or inhibited, for the manufacture of a medicament. Accordingly, the present invention provides a method of treating a subject with a population of hypoxia-resistant immune cells wherein Drp1 expression and/or activity is directly reduced or inhibited, or with a pharmaceutical composition comprising said population of hypoxia-resistant immune cells.

“Modified” as used herein in the context of “modified (optionally redirected) immune cells” or “a population of modified (optionally redirected) immune cells” indicates respectively immune cells or a population of immune cells wherein Drp1 expression and/or activity is reduced/inhibited. Evidently, any reference herein in any embodiment, aspect, or example to immune cells or a population of immune cells having a decreased Drp1 expression and/or activity are to be understood as being respectively modified immune cells or a population of modified immune cells. A skilled person appreciates that reference to modified immune cells and a population of modified immune cells does not imply that the Drp1 modification is the only modification when compared to naturally occurring immune cells, but instead indicates that the immune cells have at least a Drp1 modification. Thus, the immune cells of the present invention are modified (and optionally redirected) immune cells comprising a modification directly affecting Drp1 expression and/or activity. Yet alternatively worded, the immune cells of the present invention are modified to contain a direct modification of Drp1 expression or activity. The modification, preferably a reduction or inhibition, can have a transient or permanent character. Hence, modified immune cells may be further manipulated to be “modified and redirected” immune cells, wherein “modified” relates to the Drp1 manipulation and “redirected” to any further modification to improve the cells.

The term “hypoxia-resistant immune cells” as used herein is indicative for immune cells that are characterised by an increased resistance to hypoxic conditions compared to an unmodified immune cell. When a normal cell comes under hypoxic conditions, this will cause ATP levels to drop, cellular functions are interrupted and eventually the cells die. The hypoxia resistance of the cells of the present invention can be permanent or transitory as a result of a permanent or transitory modification of Drp1 expression and/or activity in these cells. However, as detailed below, preferably the hypoxia resistance of these cells is permanent.

It is to be appreciated that unless otherwise indicated, reference to “a population of cells” refers to an enriched or purified population of cells, but wherein the population of cells are not required to be in a pure form. Hence, “a population of immune cells” as used herein should be interpreted as “a substantially pure population of cells”. Optionally, the population of cells comprises at least about 50%, preferably at least about 60%, preferably at least about 70%, preferably at least about 80%, preferably at least about 90%, more preferably more than 90% cells that are (optionally redirected) immune cells characterized by a reduced or inhibited Drp1 expression and/or activity. Optionally, the population of cells comprises less than about 50%, preferably less than about 40%, preferably less than about 30%, preferably less than about 20%, more preferably less than about 10% cells that are not the (optionally redirected) immune cells characterized by a reduced or inhibited Drp1 expression and/or activity.

A skilled person readily appreciates that the expressions “hypoxia-resistant immune cells” may be interchangeably used with the expressions such as “immune cells having improved hypoxia resistance” and “immune cells having reduced or inhibited Drp1 expression and/or activity” since the hypoxia resistance is the effect achieved by Drp1 reduction or inhibition. The terms “hypoxia” and “hypoxic conditions” as used herein refer to a state of a tissue wherein oxygen levels are decreased when compared to “physoxia” levels (i.e. “tissue normoxia” or “normoxia”; oxygenation in healthy tissues). A distinction is to be made between a condition wherein the body of the subject as a whole (generalised hypoxia) or a region or tissue of the body (tissue hypoxia) is deprived of adequate oxygen supply. Hypoxia typically originates from a situation wherein oxygen demand exceeds oxygen supply in said tissue. In the context of the present disclosure, the terms typically refers to tissue hypoxia, more particularly certain regions in a tumour microenvironment (or bone marrow). A skilled person appreciates that normal oxygenation levels in tissues vary widely between different organs (and are by means of illustration described in Muz et al., The role of hypoxia in cancer progression, angiogenesis, metastasis, and resistance to therapy, Hypoxia (Auckl), 2015). A complete deprivation of oxygen supply is termed “anoxia”.

Hypoxia triggers a complex series of responses in tumour cells, including the activation of hypoxia-inducible factor 1 (HIF-1), which is a transcription factor that regulates the expression of genes involved in adaptation to low oxygen conditions. HIF-1 regulates the expression of genes involved in many processes, including but not limited to glucose metabolism, angiogenesis, cell proliferation, apoptosis, immune evasion, and therapy resistance.

One of the consequences of hypoxia is the shift in tumour cell metabolism from oxidative phosphorylation to glycolysis, even in the presence of oxygen, a phenomenon known as the Warburg effect. This switch to glycolysis provides the tumour cells with energy and allows them to generate metabolites that promote tumour growth and invasion. Hypoxia also promotes angiogenesis, the process of new blood vessel formation, through the upregulation of pro-angiogenic factors such as vascular endothelial growth factor (VEGF) and angiopoietin-2 (Ang-2). This process is crucial for the survival and growth of the tumour because it provides the tumour cells with nutrients and oxygen. However, the newly formed blood vessels are often leaky and abnormal, leading to further hypoxia and the accumulation of waste products in the tumour microenvironment.

In addition, hypoxia can alter the immune response by suppressing the activity of anti-tumoral immune cells and promoting the recruitment of immunosuppressive cells such as regulatory T cells (Tregs) and myeloid-derived suppressor cells (MDSCs). Particularly, in anti-tumoral immune cells hypoxia results in both overlapping and distinct effects depending on the specific immune cell type. Hypoxia promotes the immunosuppressive immune cell types while impeding the antitumoral immune cell types by impacting for example activity, migration, and survival. This creates an immunosuppressive microenvironment that promotes tumour growth and metastasis.

The hypoxia-resistance may present itself in distinct manners, such as but not limited to improved survival rates of the immune cells in hypoxic conditions, and/or improved potency of the immune cells in hypoxic conditions. Typically, the hypoxia resistance of the immune cells is apparent from an increased survival rate when compared to non-treated or non-modified immune cells upon exposure to hypoxic conditions. A person of ordinary skill in the art is capable of determining whether a given immune cell is characterized by a certain hypoxia-resistance beyond the degree of a reference immune cell and may use any suitable method described in the art to arrive at such a conclusion. By means of illustration and not limitation, a suitable assay for comparing hypoxia resistance vis-à-vis a reference cell or reference population of cells may comprise culturing both cell types or cell cultures for an equal amount of time in hypoxic conditions and in a further step evaluating the viability and/or functionality of both cell types or cell populations. Suitable methods include but are not limited to readouts based on the use of trypan blue and/or automatic cell counting.

It is well within the capacities of a skilled person to determine suitable reference cell types or cell populations when evaluating hypoxia-resistance of a cell or cell population of interest. The most evident reference cell in the context of the present invention is a cell that shares an (essential) identity on the genomic, transcriptomic, and proteomic levels with the subject cells, with any differences being those that may be the result of the Drp1 expression level manipulation, or downstream effects of said manipulation. Hence, a suitable reference cell for the immune cells subject of the present invention is a cell of the same type not comprising said modification of Drp1 expression and/or activity. Alternative expressions appropriate to define a reference cell in the present context include without limitation “an immune cell of the same type with normal (i.e. “non-modified”, “unaltered”, “wild type”, “as naturally occurring”, etc.) Drp1 expression”, “a cell corresponding to the immune cell subject of the present invention but wherein Drp1 expression and/or activity is not modified/manipulated.

In addition, it has been reported in the art that hypoxic conditions lead to aberrant mitochondrial fission in cells such as immune cells. “Mitochondrial fission” is a molecular phenomenon that has been described in detail and is therefore known to a person skilled in the art. In brief, mitochondrial fission indicates the molecular process wherein mitochondria divide into separate mitochondria. By formation of smaller mitochondria, mitochondrial fission allows a more efficient redistribution of these smaller sized organelles to energy-demanding regions. In addition, mitochondrial fission contributes to quality control by enabling the removal of damaged mitochondria as it can isolate impaired mitochondria to be eliminated by mitophagy. Therefore, mitochondrial fission is essential for mitochondrial distribution and homeostasis. In conditions of general health, mitochondrial fission is balanced by mitochondrial fusion (i.e. fusing of separate mitochondria into one larger mitochondrion) to anticipate to the metabolic needs of the cells (Al Ojaimi et al., Mitochondrial Fission and Fusion: Molecular Mechanisms, Biological Functions, and Related Disorders, Membranes (Basel), 2022). Mitochondrial fission is ensured by an interplay of different proteins.

As used throughout the present disclosure, the term “immune cells” refers to any cells of the innate and acquired immune system, and therefore includes neutrophils, eosinophils, basophils, monocytes, macrophages, dendritic cells, lymphocytes including B cells, T-cells, and natural killer cells. The term “immune cells” also encompasses immune cells that are further manipulated such as but not limited to redirected immune cells as discussed extensively further in this specification. Consequently, the term “immune cells” also encompasses primary, activated and/or expanded T cells, NK cells and other immune cell types, or immortalized cell lines thereof. The term “immune response” refers to any detectable response to a particular substance (such as an antigen) by the immune system of a host mammal, such as innate immune responses, cell-mediated immune responses, and humoral immune responses.

The term “cell population” as used herein refers generally to a group (i.e. a plurality) of cells and also encompasses a group of redirected cells. A cell population may consist of cells having a common phenotype or consists of cells wherein at least a portion thereof have a common phenotype. Cells are generally considered to have a common phenotype when they are substantially similar or identical in one or more demonstrable characteristics, including but not limited to morphological appearance, the presence, absence or level of expression of particular cellular components or products, activity of certain biochemical pathways, proliferation capacity and/or kinetics, differentiation potential and/or response to differentiation signals or behavior during in vitro cultivation (e.g., adherence, non-adherence, monolayer growth, proliferation kinetics, or the like). Such demonstrable characteristics may therefore define a cell population or a fraction thereof. The cell populations described herein may be heterogenous or (essentially or substantially) homogenous. “Heterogenous” in the present context indicates a cell population comprising two or more cells or fractions of cells not having a common phenotype (i.e. a cell population comprising cells of two or more different cell types). When a cell population is said to be “homogeneous”, it consists of cells having a common phenotype. In accordance with the generally accepted definition in the art, a cell population is considered to be “essentially homogeneous” comprises a substantial majority of cells having a common phenotype or biomarker signature. Optionally, an “essentially homogeneous” cell population may comprise at least about 75%, e.g., at least about 80%, preferably at least about 85%, preferably at least about 90%, preferably at least about 95%, more preferably at least about 99% of cells having a common phenotype. Preferred types of immune cells are described in detail further throughout the present specification.

The present inventors have identified advantageous properties of immune cells wherein Drp1 expression is directly reduced or inhibited. These advantageous properties are of particular interest for introduction into redirected immune cells.

“Drp1” as used in the present disclosure refers to “dynamin-1-like protein” or “dynamin-related protein” is a protein that in humans is encoded by the DNM1L gene located on chromosome 12 (12p11.21). Throughout the art, alternative annotations of Drp1 include DNM1L, DLP1, DRP1, DVLP, DYMPLE, EMPF, HDYNIV, dynamin 1-like, dynamin 1 like, EMPF1, and OPA5. While generally and in accordance with conventional nomenclature “DRP1” refers to the gene and “Drp1” refers to the protein, references to either one of the abbreviations in the context of the present invention can be used interchangeably since the findings of the inventors are applicable to both the transcript level and the protein level. Drp1 is a GTPase involved in the regulation of mitochondrial fission and commonly classified as a member of the dynamin protein superfamily (DSP). Drp1 is reported to mediate membrane fission through oligomerization into membrane-associated tubular structures that wrap around the scission site to constrict and sever the mitochondrial membrane through a GTP hydrolysis-dependent mechanism.

The human DRP1 gene is annotated and available under NCBI Genbank (www.ncbi.nlm.nih.gov/gene/) Gene ID: 10059. The human Drp1 canonical (i.e. reference) protein sequence, also sometimes referred to as DNM1L, is annotated and available under Uniprot (www.uniprot.org) accession number 000429-1. To date, nine isoforms have been reported, each of which are also available under Uniprot accession numbers O00429-2, O00429-3, O00429-4, O00429-5, O00429-6, O00429-7, O00429-8, and O00429-9. Unless explicitly indicated otherwise, any reference to Drp1 encompasses both the canonical product and any isoforms. Any references to certain proteins or genes throughout the present disclosure indicate human proteins or human genes unless explicitly stated otherwise.

In the context of the present disclosure, the terms “reduction” and “inhibition” are interchangeably used and indicate a diminishment of a certain value of a parameter when compared to a value of the same parameter at an earlier point in time. The reduction may be partially or complete. Particularly, reference to a reduction or inhibition of an expression (level) indicates that a certain gene and/or its gene product is expressed at a diminished amount (value) when compared to the expression (level) of the same gene and/or gene product at an earlier point in time. When references are made in the present disclosure to a reduced expression level and/or activity of Drp1, this indicates a diminishment of respectively Drp1 expression and/or activity in (an) immune cell(s) when comparing with a control. The comparison with the control may be the comparison between a first point in time and a further point in time, wherein the earlier point in time is a timepoint prior to treatment of said cell(s) with a Drp1 inhibitor. Numerous examples of such inhibitors are given throughout the present disclosure. Optionally, the Drp1 expression and/or activity is reduced by at least about 10%, preferably by at least about 20%, preferably by at least about 30%, preferably by at least about 40%, by at least about 50%, preferably by at least about 60%, preferably by at least about 70%, preferably by at least about 80%, preferably by at least about 90%, most preferably by more than 90% when compared to point in time prior to contacting the immune cells with a Drp1 inhibitor. Optionally, the Drp1 expression level is from about 5% to about 95%, preferably from about 10% to about 90%, preferably from about 20% to about 80%, preferably from about 30% to about 70%, preferably from about 40% to about 60% of the Drp1 expression level of said immune cells prior to contacting the cells with a Drp1 inhibitor. Optionally, the Drp1 activity is from about 5% to about 95%, preferably from about 10% to about 90%, preferably from about 20% to about 80%, preferably from about 30% to about 70%, preferably from about 40% to about 60% of the Drp1 activity of said immune cells prior to contacting the cells with a Drp1 inhibitor.

The reduced expression level or activity may additionally or alternatively also be determined by comparison to respectively a baseline expression level or activity which is a representative expression level or activity for the particular kind of immune cell in absence of a Drp1 inhibitor or in absence of any condition that may impact the Drp1 expression level. By means of illustration and not limitation, such a baseline expression level may be obtained by averaging the Drp1 expression level of a representative number of said immune cells, or may be considered the median Drp1 expression level of a representative number of said immune cells. Similarly, a baseline expression level may be obtained by averaging the Drp1 activity of a representative number of said immune cells, or may be considered the median Drp1 activity of a representative number of said immune cells. A person skilled in the art is capable of defining a “representative number” of cells for the purpose of establishing a baseline Drp1 expression level and/or activity. Hence, optionally the Drp1 expression and/or activity is reduced by at least about 10%, preferably by at least about 20%, preferably by at least about 30%, preferably by at least about 40%, by at least about 50%, preferably by at least about 60%, preferably by at least about 70%, preferably by at least about 80%, preferably by at least about 90%, most preferably by more than 90% when compared a baseline Drp1 expression level. Optionally, the Drp1 expression level and/or activity is from about 5% to about 95%, preferably from about 10% to about 90%, preferably from about 20% to about 80%, preferably from about 30% to about 70%, preferably from about 40% to about 60% of the Drp1 baseline expression level and/or baseline activity.

The reduced Drp1 expression level may be determined by the absolute determination of the Drp1 expression level. Similarly, the reduced Drp1 activity may be determined by any functional assay. In particular embodiments the residual expression or activity of Drp1 cannot be detected by methods described in the art. In such embodiments, an essentially complete, or complete reduction (inhibition) of Drp1 is obtained. Said reduction or inhibition of the Drp1 expression level results in an improved hypoxia-resistance.

Optionally, the determination of the Drp1 expression level and/or activity may be ensured by normalising an obtained expression value to the expression level of one or more housekeeper genes. The expression level of the one or more housekeeper genes are preferably measured in the same biological sample. “Housekeeping genes” are genes that are characterised by a constant, or essentially constant expression level in a group of cells or tissues. The term is well known to a person skilled in molecular biology and a skilled person thus appreciates that reference thereto implies a reference to one or more genes having uniform or near uniform expression levels with low variance between different samples, said samples optionally being subjected to different conditions and/or treatments. A skilled person is aware of repositories compiling housekeeping genes such as but not limited to the “Housekeeping and Reference Transcript Atlas” (Hounkpe et al., HRT Atlas v1.0 database: redefining human and mouse housekeeping genes and candidate reference transcripts by mining massive RNA-seq datasets, Nucleic Acids Res, 2021). Cellular localisation and function of genes are not particularly limiting to act as a housekeeping gene for normalisation of gene expression levels, as the suitability is solely determined by their (near) constant expression levels. Hence, suitable housekeeping genes may be identified in any of the following non-limiting gene categories: genes regulating gene expression, genes involved in metabolism, genes encoding structural cellular components, genes encoding surface proteins, kinase genes, signalling genes, etc.

A reduced Drp1 expression level may be limited to a reduction in Drp1 protein expression level, but may equally in addition encompass and/or be determined by a reduction in Drp1 transcript level (i.e. RNA level).

The term “contacting” as used herein indicates a deliberate and controlled interaction or engagement between two or more substances, in the present specification typically a controlled interaction or engagement between immune cells and a Drp1 inhibitor. Said “interaction” may encompass various processes, such as the introduction of cells into a specific growth medium, the exposure of cells to specific molecules, or the manipulation of environmental conditions. A person of ordinary skill in the art appreciates that “contacting” is a routinely used expression in a context of cell cultivation and manipulation.

The application envisages the use of the hypoxia-resistant immune cells of the invention in the treatment and/or prevention of disease in a subject.

Preferred subjects within the context of the present disclosure are human subjects. Nevertheless, terms such as “subject”, “patient”, or “individual” which are used interchangeably herein may refer to animals, preferably warm-blooded animals, more preferably vertebrates, and even more preferably mammals specifically including humans and non-human mammals. The term “mammals”, or “mammalian subjects” refers to any animal classified as such and include, but are not limited to, humans, domestic animals, commercial animals, farm animals, zoo animals, sport animals, pet and experimental animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Preferred subjects are human subjects including all genders and all age categories thereof. Both adult subjects, newborn subjects, and foetuses are intended to be covered by the term “subject”.

The population of hypoxia-resistant immune cells are envisaged by the present disclosure for use in treating neoplastic diseases such as cancer. Further envisaged is the use of hypoxia-resistant immune cells wherein Drp1 expression is directly reduced or inhibited for the manufacture of a medicament for the treatment of cancer preferably a cancer that is characterised by, or associated with, the presence of a hypoxic tumour microenvironment. Optionally, the hypoxia-resistant immune cells are used in a ratio to the target neoplastic cells in a 10:1 to 1:10 ratio, preferably in a 2:1 to 1:7 ratio, more preferably in a 1:1 to 1:5 ratio.

The term “neoplastic disease” generally refers to any disease or disorder characterized by neoplastic cell growth and proliferation, whether benign (not invading surrounding normal tissues, not forming metastases), pre-malignant (pre-cancerous), or malignant (invading adjacent tissues and capable of producing metastases). The term neoplastic disease generally includes all transformed cells and tissues and all cancerous cells and tissues. Neoplastic diseases or disorders include, but are not limited to abnormal cell growth, benign tumours, premalignant or precancerous lesions, malignant tumours, and cancer.

As used herein, the term “cancer” refers to a malignant neoplasm characterized by deregulated or unregulated cell growth. The term “cancer” includes primary malignant cells or tumours (e.g., those whose cells have not migrated to sites in the subject's body other than the site of the original malignancy or tumour) and secondary malignant cells or tumours (e.g., those arising from metastasis, the migration of malignant cells or tumour cells to secondary sites that are different from the site of the original tumour). The term “metastatic” or “metastasis” generally refers to the spread of a cancer from one organ or tissue to another non-adjacent organ or tissue. The occurrence of the neoplastic disease in the other non-adjacent organ or tissue is referred to as metastasis.

Examples of cancer types include but are not limited to carcinoma, sarcoma, lymphoma, blastoma, and leukaemia or lymphoid malignancies. More particular examples of such cancer types include without limitation: squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as CNS cancer, melanoma, head and neck cancer, bone cancer, bone marrow cancer, duodenum cancer, oesophageal cancer, thyroid cancer, or haematological malignancy/cancer (e.g. lymphoma, myeloma, and leukaemia).

In particular embodiments, the cancer types which are envisaged by the present disclosure are characterised by the presence of a hypoxic (tumour) (micro)environment. The expression “(tumour) microenvironment” or “(T)ME” is known to a skilled person and refers to the resulting microenvironment generated by cancer cells through inducing molecular, cellular, and physical changes within the tissue of the subject and can be either an internal region of a tumour (e.g. a solid tumour) or a microenvironment associated with the cancer cells (e.g. hypoxic bone marrow environment in leukaemia). The TME of solid tumours is frequently hypoxic owing to defects in vasculature coupled to increased local oxygen demand. In particular embodiments, the solid tumour (micro) environment is determined to be particularly hypoxic as a result of one or more of the tumour stage, proliferation rate, vasculature, location, etc.

In preferred embodiments, the population of hypoxia-resistant immune cells is used for treating a malignancy (i.e. cancer). Hence, provided is a method for treating cancer, in particular haematological malignancies such as a solid tumour or leukaemia, comprising administering to a subject in need thereof a hypoxia-resistant immune cell wherein Drp1 expression and/or activity is directly reduced or inhibited. Further envisaged is the use of a hypoxia-resistant immune cell wherein Drp1 expression and/or activity is directly reduced or inhibited for the manufacture of a medicament for the treatment of cancer, such as a solid tumour or leukaemia.

As used herein, the terms “tumour”, “solid tumour” or “tumour tissue” each refer to an abnormal mass of tissue that results from excessive cell division. A tumour or tumour tissue comprises tumour cells which are neoplastic cells with abnormal growth properties and no useful bodily function. A tumour or tumour tissue may generally further comprise tumour-associated non-tumour cells, e.g., vascular cells which form blood vessels to supply the tumour or tumour tissue. Non-tumour cells may be induced to replicate and develop by tumour cells such as but not limited to the induction of angiogenesis in a tumour or tumour tissue. Solid tumour cancers that are envisaged by the inventors include without limitation: adrenal gland tumours, anal cancers, appendix cancers, bladder cancers, bone cancers, brain cancers, breast cancers, cervical cancers, bile duct cancers, bone cancers, colorectal cancers, endometrial cancers, oesophageal cancers, eye cancers, gallbladder cancers, gastrointestinal stromal cancers, head cancers, neck cancers, kidney cancers, soft tissue cancers, liver cancers, lung cancers, lymphomas, skin cancers (melanomas), mesotheliomas (cancers of the lining of the lungs or abdomen), bone marrow cancers, nasal cavity cancers, paranasal sinus cancers, neuroblastomas, ovarian cancers, pancreatic cancers, penile cancers, peritoneal cancers, prostate cancers, rectal cancers, kidney cancers, sarcomas, non-melanoma skin cancers, small intestine cancers, stomach cancers, testicular cancers, thymomas, thyroid cancers, uterine sarcomas, vaginal cancers, and vulvar cancers.

A further preferred malignancy (i.e. cancer) type characterized by the presence of hypoxia is leukaemia, as oxygen levels in bone marrow can drop to 1%. More preferably, the leukaemia is selected from the group consisting of: acute myeloid leukaemia (AML), chronic myeloid leukaemia (CML), acute lymphocytic leukaemia (ALL), chronic lymphocytic leukaemia (CLL), hairy cell leukemia (HCL), large granular lymphocytic leukemia (LGLL), mixed phenotype acute leukemia (MPAL), myelodysplastic syndrome (MDS), myeloproliferative neoplasms (MPN), and T cell prolymphocytic leukemia (T-PLL). Yet more preferably, the leukaemia is acute myeloid leukaemia (AML). It is to be understood that acute myeloid leukaemia (AML) is a highly heterogeneous hematologic malignancy that results from a wide range of alterations of myeloid precursors responsible for the uncontrolled proliferation, block of differentiation, and escape from apoptosis. The resulting expansion of leukemic-initiating cells induces bone marrow (BM) failure, leading to a loss of normal hematopoietic functions. Hence, the hypoxia-resistant immune cells are particularly suited for treating subjects that are characterized by a hypoxic bone marrow microenvironment.

Alternative preferred cancer types (i.e. tumour types) in the context of the present disclosure are selected from the group consisting of: liver cancers (liver tumours), pancreatic cancers (pancreatic tumours), colorectal cancers (colorectal tumours), breast cancers (breast tumours), head tumours, neck tumours, and lung tumours. More preferably selected from the group consisting of pancreatic cancers (pancreatic tumours), colorectal cancers (colorectal tumours), head tumours, and neck tumours.

The population of hypoxia-resistant cells subject of the present disclosure may be used as an autologous cell therapy or alternatively as an allogenic therapy, depending on the source of the cells and the subject that is being treated. A skilled person is familiar with both therapy strategies and their differences. In brief, “autologous cell therapy” refers to a treatment wherein cells of the subject are extracted from the body prior to manipulation of the extracted cells in vitro and finally reinsertion of the manipulated cells into the subject. In contrast, “allogenic cell therapy” refers to a treatment wherein cells are administered to a subject that have an origin which is not the subject. Preferably, the invention envisages a population of hypoxia-resistant immune cells wherein Drp1 expression is directly reduced or inhibited for use in allogenic cell therapy. The invention is also directed to the use of a population of hypoxia-resistant immune cells wherein Drp1 expression is directly reduced or inhibited, for use in allogenic cell therapy. In addition, the invention is directed to a method of allogenic cell therapy, said method comprising administration of hypoxia-resistant immune cells wherein Drp1 expression is directly reduced or inhibited to a subject.

Hence, provided is a method for treating a neoplastic disease such as cancer, comprising administering to a subject in need thereof a hypoxia-resistant immune cell wherein Drp1 expression and/or activity is directly reduced or inhibited, or a pharmaceutical composition comprising said cells. The terms “treatment” or “treat” as used herein encompass both the therapeutic treatment of an already developed disease or condition, such as the therapy of an already developed tumour characterised by a hypoxic tumour microenvironment, as well as measures to inhibit a tumour characterised by a hypoxic tumour microenvironment to develop (i.e. an early use of the hypoxia-resistant immune cells as described herein to prevent the development of a hypoxic tumour microenvironment). The aim of the hypoxia-resistant cells described herein is to prevent or lessen the development of a cancer (such as leukaemia or a solid tumour). Beneficial or desired clinical results may include, without limitation, alleviation of one or more symptoms, diminishment of the extent of disease (e.g. a reduction in tumour size), stabilized (i.e., not worsening) state of disease (e.g. inhibition of tumour growth), delay or slowing of disease progression (e.g. slowing down tumour growth), amelioration or palliation of the disease state, and the like. “Treatment” can also mean prolonging survival of the subject as compared to expected survival of said subject if not receiving treatment.

As used herein, the terms “therapeutic treatment” or “therapy” and the like, refer to treatments wherein the object, or goal, is to change a subjects body or a part of a subjects body from an undesired physiological state, disease or disorder, such as the presence of malignant cells, to a desired state, such as a less severe state (e.g., amelioration or palliation), or even back to its normal, healthy state (e.g., restoring the health, the physical integrity and the physical well-being of a subject), to keep it (i.e., not worsening) at said undesired physiological status (e.g., stabilization), or slow down progression to a more severe or worse state compared to said undesired physiological change or disorder. Measurable lessening includes any statistically significant decline in a measurable marker, symptom, number of malignant cells, a reduction in tumour size, and any combination thereof. Generally, the terms encompass both curative treatments and treatments directed to reduce symptoms and/or slow progression and/or stabilize the disease. A skilled person is aware that in order to achieve an effective therapeutic treatment, a therapeutically effective dose needs to be administered to said subject. The hypoxia-resistant cells subject of the invention may also be used to prevent disease progression, or even prevent the manifestation of clinical symptoms of the disease. “Prevention” or “prevent” as used in the context of the invention refers to an aversion of manifestation of a disease image in a subject, i.e. the establishment of preventive measures or prophylactic measures. Preventive treatment refers to treatments wherein the object is to avoid a subject's body or an element thereof to show clinical symptoms of an undesired physiological change or disorder. A skilled person is aware that in order to achieve an effective therapeutic treatment, a prophylactically effective dose needs to be administered to said subject.

The term “therapeutically effective dose” or “therapeutically effective amount” as used herein refers to an amount of hypoxia-resistant immune cells as taught herein, that when administered brings about a positive therapeutic response with respect to treatment of a subject suffering from a disease, e.g. a patient having been selected (e.g. diagnosed) to have or a certain disease. In certain embodiments, the patient is diagnosed with cancer, such as but not limited to leukaemia or a solid tumour cancer. A skilled person further understands that the required dosage or amount that is needed to arrive at a therapeutically effective dose needs to be assessed on a case-by-case and subject-to-subject basis. An effective amount is also such an amount in which any toxic or detrimental effects of the treatment are outweighed by the therapeutically beneficial effects. It is standard practice to adapt a dosage to a certain individual to obtain an optimal, i.e. ideal effect or response. Hence, a plethora of parameters may be assessed when determining an optimal dosage, or dosage schedule and include but are by no means limited to the nature and degree of the disease to be treated, gender of the subject, subject age, body weight, other medical indications, nutrition, mode of administration, metabolic state, interference or influence of efficacy by other pharmaceutically active ingredients, etc. Furthermore each cancer or tumour (and therefore each subject) may have a certain intrinsic degree of responsiveness to the hypoxia-resistant immune cells that are used. In the present context of cancer or tumour, an effective amount of the hypoxia-resistant immune cells described herein may have the effect in reducing the number (i.e. amount) of cancer cells; reducing the tumour size; inhibiting (i.e. slow to some extent or desirably stop) cancer cell infiltration into peripheral organs; inhibit (i.e. slow to some extent and desirably stop) tumour metastasis; inhibiting to some extent tumour growth; and/or relieving to some extent one or more of the symptoms associated with the cancer or tumour.

The hypoxia-resistant immune cells are typically administered to a subject that is diagnosed to have cancer, preferably a subject that is diagnosed to have a haematological malignancy (e.g. leukaemia) or a solid tumour cancer. The means used for establishing the cancer diagnosis is not limiting for the invention and include without limitation any imaging means (optionally including a step of taking a biopsy from the subject) and molecular profiling assays. The term “diagnosing” as used herein well known to a person skilled in the art and is indicative for a process of recognizing, deciding on or concluding on a cancer disease in a subject on the basis of symptoms and signs and/or from results of various diagnostic procedures (such as, for example, from knowing the presence, absence and/or quantity of one or more biomarkers of or clinical symptoms characteristic for the cancer, or a particular cancer type).

Use of the hypoxia-resistant cells subject of the invention may have a positive impact on the prognosis of the cancer that was earlier diagnosed in the subject. “Prognosticating” in the context of the invention is indicative for anticipation on the progression of the cancer and the prospect (e.g. the probability, duration, and/or extent) of recovery, and/or the severity of the cancer. The term “a good prognosis of” generally encompass anticipation of a satisfactory partial or complete recovery from a diagnosed cancer. Alternatively, the term may encompass anticipation of not further worsening or aggravating of such, preferably within a given time period. The term “a poor prognosis of” the disease or condition typically encompass an anticipation of a substandard recovery and/or unsatisfactorily slow recovery, or no recovery at all, or further worsening of said cancer, or any clinical manifestation associated with said cancer. Optionally, the subject may be a subject wherein conventional anti-cancer therapies such as radiotherapy and/or chemotherapy have been found to be inadequate to treat the cancer, or in subjects having a cancer which is observed to be, considered to be, or suspected to be resistant to conventional anti-cancer therapies such as radiotherapy and/or chemotherapy.

The uses and treatments described herein may be supplemented by monitoring the effects thereof. A skilled person is aware that the monitoring of a disease or a condition may allow to predict the occurrence of the disease or condition, or to monitor the progression, aggravation, alleviation or recurrence of the disease or condition, or response to treatment or to other external or internal factors, situations or stressors, etc. Such monitoring may assist in for example the decision making whether a patient may be discharged from a controlled clinical or health practice environment, needs a change in treatment or therapy, or requires (further) hospitalization or medical intervention.

As indicated throughout the present disclosure, the immune cells of the present invention, are immune cells which are of interest in the treatment of cancer and further are modified to ensure hypoxia-resistance characteristics by reduction or inhibition of Drp1 expression (and/or Drp1 activity, i.e. function).

Preferably, the hypoxia-resistant immune cells described herein are cells wherein Drp1 expression and/or activity is permanently reduced or inhibited (i.e. treated with a Drp1 inhibitor that has lasting effects on Drp1 expression). Preferably, the hypoxia-resistant immune cells are Drp1 knock out cells, i.e. the Drp1 expression is inhibited as a result of one or more modifications to the Drp1 gene. Thus, in particular embodiments, the immune cells of the present invention are modified and/or genetically engineered, preferably they comprise a modified Drp1 gene which prevents expression of a functional Drp1 protein.

Alternatively, the immune cells are treated by a Drp1 inhibitor which mediates its effect in a conditional manner on the immune cells. By the term “conditional manner” as used herein is meant that the Drp1 inhibitor only exerts its effects when certain conditions are fulfilled, such as the presence of the inhibitor or an activation signal. Different examples of Drp1 inhibitors are provided below.

In particular embodiments, the Drp1 expression of the immune cells is reduced compared to the same immune cell which has not been contacted with the Drp1 inhibitor.

Preferably the Drp1 expression is decreased by at least 2-fold, at least 4-fold, at least 6-fold, at least 8-fold, at least 10-fold in a cell or cellular system when compared to a control or compared to the expression in absence of Drp1 inhibitor or before addition of the Drp1 inhibitor. In certain embodiments, the expression of Drp1 protein is reduced by at least about 30%, at least about 40%, at least about 50%, at least about 60%, at least about 65%, at least about 70%, at least about 75%, at least about 80%, at least about 85%, at least about 90%, at least about 95% in the immune cells when compared to a control or compared to the expression in absence of Drp1 inhibitor or before contacting the cells with the Drp1 inhibitor. Examples of Drp1 inhibitors which can ensure (preferably permanently) reduced expression levels include genomic engineering methods described herein. Methods for determining reduced expression levels of Drp1 are known in the art and include but are not limited to Northern blotting, quantitative polymerase chain reaction (qPCR), DNA microarray, and RNA-Seq. However, it will be understood by the skilled person that in the context of the present invention, reduced gene expression leads to reduced protein levels, such that reduced expression can also be measured at the protein level, such as methods based on immunological detection known in the art.

In particular embodiments, the Drp1 activity of the immune cells is reduced compared to the same immune cell which has not been contacted with the Drp1 inhibitor.

Preferably the Drp1 activity is decreased by at least 2-fold, at least 4-fold, at least 6-fold, at least 8-fold, at least 10-fold in a cell or cellular system when compared to a control or compared to the Drp1 activity in the cell in absence of Drp1 inhibitor or before addition of the Drp1 inhibitor. In certain embodiments, the activity of Drp1 protein is reduced by at least about 30%, at least about 40%, at least about 50%, at least about 60%, at least about 65%, at least about 70%, at least about 75%, at least about 80%, at least about 85%, at least about 90%, at least about 95% in the immune cells when compared to a control or compared to the Drp1 activity in absence of Drp1 inhibitor or before addition of the Drp1 inhibitor.

In certain embodiments, said cells may be characterised by a Drp1-616 phosphorylation signal that is at least 10%, preferably at least 20%, more preferably at least 30%, more preferably at least 40%, more preferably at least 50, most preferably at least 60% reduced when compared to immune cells corresponding to those used as starting cell type for reducing the Drp1 expression level. A skilled person appreciates that Drp1-616 phosphorylation is a suitable means to evaluate Drp1 expression (and in some cases also activation).

As detailed above, the reduced expression and/or activity of Drp1 in the immune cells of the present invention can also be measure by determining their hypoxia-resistance as this is envisaged to be a direct consequence thereof.

As detailed above, the immune cells of the present invention are typically obtained by contacting immune cells having other characteristics of interest with an inhibitor of Drp1. The terms “inhibitor of Drp1”, “Drp1 inhibitor”, and even “inhibitor” may be used interchangeably herein and refer to any agent that can be regarded to have an inhibitory effect on Drp1. The means for reducing or inhibiting Drp1 expression and/or activity are not particularly limited for the invention, and the expression may therefore be reduced by a Drp1 inhibitor that acts on the gene encoding Drp1, any Drp1 regulating genomic sequence, the Drp1 transcript (i.e. mRNA), or the Drp1 protein. Thus, the inhibitor may act directly on the Drp1 encoding sequence or on Drp1 protein. The Drp1 inhibitor is envisaged to be a “direct” inhibitor, i.e. a moiety that “directly” inhibits Drp1 expression and/or activity. “Directly” in this context indicates that the inhibitor acts directly on the Drp1 protein, transcript, or genomic sequence and indirectly be means of interaction with for example a Drp1-binding protein. Hence, terms used throughout the present specification such as “direct inhibition” and “direct reduction” imply that a “specific” inhibitor is used, i.e. a molecule or compound that selectively (i.e. with increased preference) binds to Drp1 protein, Drp1 transcripts, or the Drp1 genomic sequence (i.e. the DNM1L gene), preventing it from functioning normally. “Encoding” is to be interpreted according to the common interpretation in the art and therefore indicates that a nucleic acid sequence or part(s) thereof corresponds, by virtue of the genetic code of an organism in question to a particular amino acid sequence, e.g. the amino acid sequence of one or more desired proteins or polypeptides, or to another nucleic acid sequence in a template-transcription product (e.g. RNA or RNA analogue) relationship.

Optionally, the Drp1 inhibitor is an inhibitor selected from the group consisting of: chemical inhibitors, binding proteins, gene editing systems, or antisense agents. Preferred means to reduce or inhibit Drp1 expression are moieties resulting in an irreversible (i.e. permanent) reduction or inhibition of Drp1. The term “inhibitor” broadly refers to any chemical (e.g., inorganic or organic), biochemical or biological substance, molecule or macromolecule (e.g., biological macromolecule), a combination or mixture thereof. Non-limiting examples of inhibitors include nucleic acids, oligonucleotides, ribozymes, peptides, polypeptides, proteins, peptidomimetics, antibodies, antibody fragments, antibody-like protein scaffolds, aptamers, photoaptamers, spiegelmers, chemical substances, preferably organic molecules, more preferably small organic molecules, lipids, carbohydrates, polysaccharides, and any combinations thereof. A skilled person is well aware that the inhibitory effect of an inhibitor may be verified by in vitro, in vitro, ex vivo, or in silico methodologies, or any combination thereof.

The term “protein” as used throughout this specification generally encompasses macromolecules comprising one or more polypeptide chains, i.e., polymeric chains of amino acid residues linked by peptide bonds. As used herein, the term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. Without limitation, protein, polypeptides or peptides can be produced recombinantly by a suitable host or host cell expression system and isolated therefrom (e.g., a suitable bacterial, yeast, fungal, plant or animal host or host cell expression system), or produced recombinantly by cell-free transcription and/or translation, or non-biological protein, polypeptide or peptide synthesis.

The term “nucleic acid” as used throughout this specification typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally occurring nucleic acids, other naturally occurring bases (e.g., xanthine, inosine, hypoxanthine) as well as chemically or biochemically modified (e.g., methylated), non-natural or derivatised bases. The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. RNA is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA). A nucleic acid can be double-stranded, partly double stranded, or single-stranded. Where single-stranded, the nucleic acid can be the sense strand or the antisense strand. In addition, nucleic acid can be circular or linear.

The term “nucleic acid” also encompasses any other nucleobase containing polymers such as nucleic acid mimetics, including, without limitation, peptide nucleic acids (PNA), peptide nucleic acids with phosphate groups (PHONA), locked nucleic acids (LNA), morpholino phosphorodiamidate-backbone nucleic acids (PMO), cyclohexene nucleic acids (CeNA), tricyclo-DNA (tcDNA), and nucleic acids having backbone sections with alkyl linkers or amino linkers (e.g. Kurreck, Antisense technologies. Improvement through novel chemical modifications, Eur J Biochem, 2003). “Alkyl” as used herein particularly encompasses lower hydrocarbon moieties, e.g., C1-C4 linear or branched, saturated or unsaturated hydrocarbon, such as methyl, ethyl, ethenyl, propyl, 1-propenyl, 2-propenyl, and isopropyl. Nucleic acids as intended herein may include naturally occurring nucleosides, modified nucleosides or mixtures thereof. A modified nucleoside may include a modified heterocyclic base, a modified sugar moiety, a modified inter-nucleoside linkage or a combination thereof.

Thus, in certain embodiments described herein the Drp1 inhibitor used for obtaining the hypoxia-resistance immune cells is selected from the group consisting of a chemical substance, an antibody, an antibody fragment, an antibody-like protein scaffold, a protein or polypeptide, a peptide, a peptidomimetic, an aptamer, a photoaptamer, a spiegelmer, and a soluble receptor, or an Antibody mimetic such as an Alphabody, a Designed Ankyrin Repeat Protein (DARPin), a Monobody, an Affibody, an Anticalin, an Avimer, a Versabody, an Uocalin, or is selected from a nucleic acid, a gene-editing system and an antisense agent. In further embodiments the inhibitor of Drp1 is selected from the group consisting of chemical substance, an antibody, an antisense agent, and a gene editing system. In certain embodiments, the inhibitor of Drp1 is selected from the group consisting of DRP1-binding molecules and Drp1 gene targeting molecules. In certain embodiments, Drp1 inhibitor specifically recognizes one or more Drp1 protein isoforms or gene isoforms. A skilled person is aware that the terms “recognizing” and “targeting” can be interchangeably used with “binding” or “hybridizing to” in this context. The term “specifically” in the context of “binding”, “hybridizing to” or “targeting” implies that the agent or inhibitor is developed to bind to, or target, a given protein or DNA/RNA sequence, without substantially binding or hybridizing to the sequence of another protein or DNA/RNA.

Chemical inhibitors of Drp1 have been described in the art and include without limitation Mdivi-1, P110 (Qi et al., J Cell Sci, 2013), and dynasore (Macia et al., Dev Cell, 2006). A particularly preferred chemical inhibitor of Drp1 in the context of the present invention for generating a population of hypoxia-resistant immune cells is Mitochondrial division inhibitor 1 (“Mdivi-1” or “Mdivi1”). Mdivi-1 has been extensively described in the art and is interchangeably indicated by synonyms including but not limited to 3-(2,4-dichloro-5-methoxyphenyl)-2-sulfanyl-4(3H)-quinazolinone. Chemical properties of Mdivi-1 are readily retrievable from public repositories such as PubChem (www.pubchem.ncbi.nlm.nih.gov; ID 3825829).

In particular embodiments, the inhibitor of Drp1 used to generate the hypoxia-resistant immune cells decreases the expression of the Drp1 protein. The reduction in expression levels may be on protein level or RNA level. Optionally, a reduction of Drp1 protein expression levels may be achieved by an interference of the inhibitor on the translation process, or a reduction of Drp1 RNA levels may be achieved by an interference of the inhibitor on the transcription process.

A skilled person may use any known technique to measure Drp1 expression levels before and/or after treatment of the immune cells with the Drp1 inhibitor. By means of guidance, a commonly used technique to measure RNA expression levels is to conduct a real-time polymerase chain reaction (RT-PCR or qPCR) experiment. RT-PCR has the ability to monitor the progress of the PCR as it occurs (i.e., in real time). There are two main methods used to perform quantitative PCR: dye-based and probe-based detection. Both methods rely on calculating the initial (zero cycle) DNA concentration by extrapolating back from a reliable fluorescent signal. Methodologies relying on RT-PCR have been described in detail in the art (Arya et al., Basic principles of real-time quantitative PCR, Expert review of molecular diagnostics, 2015). Commonly used techniques to measure protein expression levels include but are by no means limited to mass spectrometry analyses, spectrophotometric assays and enzyme-linked immunosorbent assays (ELISA). Non-limiting examples of targeted proteomics experiments include selective reaction monitoring and multiple reaction monitoring. Mass spectrometry approaches to measure protein expression levels have been extensively described in the art (Shi et al., Advances in targeted proteomics and applications to biomedical research, Proteomics, 2016). Spectrophotometric assays include UV light absorption spectroscopy, dye-based protein assays, Coomassie blue (Bradford) assays, and Lowry alkaline copper reduction assays. These, and others, have been described in the art (Noble and Bailey, Quantitation of protein, Methods in enzymology volume 463, 2009). ELISA assays suitable for protein quantitation has also been described (Parnas and Linial, Highly sensitive ELISA-based assay for quantifying protein levels in neuronal cultures, Brain research protocols, 1998).

In certain embodiments, the inhibitor of Drp1 decreases the expression of the Drp1 protein by acting on the DNA or RNA level of Drp1. Hence, in such embodiments, the inhibitor of Drp1 may be a Drp1 inhibitor acting on the DNM1L gene. Optionally, the Drp1 inhibitor inhibits the expression of Drp1 preferably by at least 2-fold, at least 4-fold, at least 6-fold, at least 8-fold, at least 10-fold in a cell or cellular system when compared to a control or compared to the expression in absence of Drp1 inhibitor or before addition of the Drp1 inhibitor. In certain embodiments, the inhibitor of Drp1 decreases the expression of Drp1 protein by at least about 30%, at least about 40%, at least about 50%, at least about 60%, at least about 65%, at least about 70%, at least about 75%, at least about 80%, at least about 85%, at least about 90%, at least about 95% in the immune cells when compared to a control or compared to the expression in absence of Drp1 inhibitor or before addition of the Drp1 inhibitor.

Examples of agents that can be generated in order to reduce the expression of a protein such as Drp1 are known in the art. The most commonly used systems are antisense molecules, RNAi and gene editing systems, each of which can be designed to specifically target the Drp1 encoding RNA or DNA sequence (i.e. the DNM1L gene sequence).

Hence, optionally the inhibitor of Drp1 used to generate the hypoxia-resistant immune cells is selected from the group consisting of an antisense agent and a gene editing system, also referred to as a Drp1 gene targeting inhibitors.

The term “antisense” generally refers to an oligonucleotide configured to specifically anneal with (hybridise to) a given sequence in a target nucleic acid, typically an mRNA. It typically comprises, consist essentially of or consist of a nucleic acid sequence that is complementary or substantially complementary to said target nucleic acid sequence. Antisense agents suitable for use herein may typically be capable of annealing with (hybridising to) the respective target nucleic acid sequences at high stringency conditions, and capable of hybridising specifically to the target under physiological conditions. Methods regarding the synthesis, manipulation and introduction into cells of antisense agents are known to the skilled person (Dias and Stein, Antisense Oligonucleotides: Basic Concepts and Mechanisms, Molecular cancer therapeutics, 2002). The sequence of an antisense agent does not need to be perfect (i.e. 100%) complementary to that of its target sequence to bind or hybridise specifically with the latter. An antisense agent may be said to be specifically hybridisable when binding of the agent to a target nucleic acid molecule interferes with the normal function of the target nucleic acid such as to attain an intended outcome (e.g. loss of utility), and there is a sufficient degree of complementarity to avoid non-specific binding of the antisense agent to non-target sequences under conditions in which specific binding is desired, i.e. under physiological conditions in the case of in vivo assays or therapeutic treatment, and in the case of in vitro assays, under conditions in which the assays are performed.

The terms “complementary” or “complementarity” as used herein with reference to nucleic acids, refer to the normal binding of single-stranded nucleic acids under permissive salt (ionic strength) and temperature conditions by base pairing, preferably Watson-Crick base pairing. By means of example, complementary Watson-Crick base pairing occurs between the bases A and T, A and U or G and C. For example, the sequence 5′-A-G-U-3′ is complementary to sequence 5′-A-C-U-3′.

In certain embodiments wherein the inhibitor of Drp1 is an antisense agent or a gene editing system, the inhibitor is directed to a portion of a nucleotide sequence encoding Drp1 in a genomic sequence of the immune cells (i.e. the DNM1L gene sequence). In alternative embodiments wherein the inhibitor of Drp1 is an antisense agent or a gene editing system, the inhibitor is directed to a portion of the Drp1 promoter sequence (i.e. the DNM1L promoter sequence). In further embodiments wherein the inhibitor of Drp1 is an antisense agent or a gene editing system, the inhibitor comprises a nucleotide sequence that has a sequence identity of at least about 65%, at least about 70%, at least about 75%, at least about 80%, at least about 85%, at least about 90%, at least about 95%, at least about 97%, at least about 99% compared to a naturally occurring genomic sequence in the DNM1L gene.

In further embodiments, the Drp1 inhibitor used to generate the hypoxia-resistant immune cells is a nucleotide sequence, such as an antisense RNA described above, wherein the nucleotide sequence has at least one terminal modification. In yet further embodiments, the one or more terminal modifications reduce degradation of the nucleotide sequence by nucleases upon introduction into the cell, organism or subject. As non-limiting example, the oligonucleotide modification may be the introduction of at least one phosphorothioate bond in the phosphate backbone of an oligo wherein a sulfur atom is substituted for a non-bridging oxygen. In certain embodiments, the at least one phosphorothioate bond is introduced at the 5′ or 3′ terminus. In certain embodiments, the at least one phosphorothioate bond is introduced internally.

In particular embodiments, the inhibitor is an RNAi, shRNA, siRNA, or miRNA. As described herein, the term “RNAi” is short for “RNA interference agent” and refers to oligonucleotide RNA sequences that provoke RNA interference by neutralizing targeted mRNA molecules and a subsequent decrease of target gene expression, in the context of the invention Drp1 gene expression. RNAi is an RNA-dependent gene silencing process which is known to be controlled by the RNA-induced silencing complex (RISC). RNAi agents are dsRNA (typically more than 200 bp) that comprise a double-stranded portion, fragment, or region of annealed oligonucleotide standards that are complementary, wherein one oligonucleotide sequence has a sequence, or a sequence corresponding to, that equals or nearly equals the sequence of (a portion of) mRNA of the target gene that is intended to be knocked down by the agent. A second strand of the RNAi agent is complementary to the target nucleotide sequence. Various RNAi agents have been described in the art and include the non-limiting examples shRNA, siRNA, and miRNA.

In particular embodiments, the Drp1 inhibitor is a gene-editing system, more particularly a gene-editing system targeting the Drp1-encoding gene (i.e. the DNM1L gene). In such embodiments, the Drp1 inhibitor reduces or inhibits Drp1 expression and/or activity by genetic modification of the Drp1 gene (i.e. DNM1L) or promoter (DNM1L promoter) sequence. A skilled person appreciates that “genetic modification” indicates an alteration of a genomic sequence, which may be a deletion, substitution, and/or insertion of one or more nucleotides. Gene editing systems are particularly preferred for generating the hypoxia-resistant immune cells of the invention and are able to generate Drp1 knockout cells in a robust manner. The term “gene-editing system” or “genome editing system” as used herein refers to a tool to induce one or more nucleic acid modifications, such as DNA or RNA modifications, into a specific DNA or RNA sequence within a cell. Gene editing systems typically make use of an agent capable of inducing a nucleic acid modification. In certain embodiments, the agent capable of inducing a nucleic acid modification is a (endo)nuclease or a variant thereof having altered or modified activity. (endo)Nucleases typically comprise programmable, sequence-specific DNA- or RNA-binding modules linked to a nonspecific DNA or RNA cleavage domain. In DNA, these nucleases create site-specific double-strand breaks at desired locations in the genome. The induced double-stranded breaks are repaired through non-homologous end-joining or homologous recombination, resulting in targeted mutations. Non-limiting examples of endonucleases are restriction enzymes, meganucleases, zinc-finger nucleases (ZFNs), transcription activator like effector nucleases (TALENs), and CRISPR-associated (Cas)-based nucleases.

Thus, in certain embodiments the Drp1 inhibitor envisaged herein are Drp1 gene (i.e. DNM1L) editing inhibitors which are optionally selected from the group essentially consisting of restriction enzymes, meganucleases, zinc finger nucleases, transcription activator like effector nucleases, or CRISPR-associated nucleases. In certain embodiments, the Drp1 inhibitor is a gene editing system that specifically cleaves a genomic sequence encoding Drp1, or a Drp1 (DNM1L) promoter sequence. The term “promoter” as defined herein is a region of DNA that initiates transcription of a particular gene and hence enables a gene to be transcribed. A promoter is recognized by RNA polymerase, which then initiates transcription. Thus, a promoter contains a DNA sequence that is either bound directly by, or is involved in the recruitment, of RNA polymerase. A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene. Promoters may be located in close proximity of the start codon of genes, in preferred embodiments on the same strand and typically upstream (5′) of the gene. Promoters may vary in size, and are preferably from about 100 to 1000 nucleotides long.

The term “restriction enzyme” as used herein can be used interchangeably with “restriction endonuclease” or “restrictase” and is indicative for a subgroup of endonucleases that cleave DNA at or in close proximity of specific recognition sites, which are commonly referred to as “restriction sites” in the art. A fragment resulting from the cutting of a DNA strand by restriction enzymes is known as a restriction fragment. Numerous restriction enzymes have been identified in the art. Methods, tools, and databases have been described in the art and are freely available to find information on both restriction enzyme activity and restriction sites (Roberts et al., REBASE—enzymes and genes for DNA restriction and modification, Nucleic Acids Research, 2007).

In certain embodiments, the Drp1 inhibitor is a meganuclease that specifically cleaves a genomic sequence encoding Drp1, or a Drp1 promoter sequence (DNM1L promoter sequence). “Meganucleases” are a group of nucleases that are characterized by a larger recognition site than standard restriction enzymes, with said recognition site typically having a length of between 12 and 40 base pairs. As a consequence, the recognition site of meganucleases is unique or near unique for any given genome. Meganucleases have been identified in a large number of organisms, inter alia in Archaea, bacteria, phages, fungi, yeast, algae, plants with unique recognition sites. Furthermore, tools to produce artificial meganucleases have been described in the art (Bartsevich, et al., Meganucleases as an efficient tool for genome engineering, Molecular Therapy, 2016). In certain embodiments the meganuclease is an intron-encoded nuclease. In certain embodiments, the Drp1 inhibitor is a ZFN that specifically cleaves a genomic sequence encoding Drp1, or a Drp1 promoter sequence (i.e. a-the DNM1L gene or DNM1L promoter).

“Zinc-finger nucleases”, commonly referred to in the art as ZFNs, are artificial restriction enzymes that comprise a zinc finger DNA binding domain fused to a DNA cleavage domain. Zinc finger domains can be engineered to target specific desired DNA sequences. This allows for a skilled person to design zinc finger nucleases that are able to target unique sequences within a given genomic sequence. A standard zinc finger DNA binding domain includes between three and six zinc finger repeats that are each capable of recognizing 9 to 18 basepairs. Diverse methods to generate zinc finger arrays are therefore known to a skilled person (Wu et al., Custom-designed zinc finger nucleases: What is Next?, Cellular and molecular life sciences, 2007). By means of guidance, an example of a suitable non-specific cleavage domain is the obligate dimeric endonuclease FokI and FokI domains with enhanced cleavage activity such as Sharkey (Guo et al., Directed evolution of an enhanced and highly efficient FokI cleavage domain for zinc finger nucleases, Journal of molecular biology, 2010). Obligate heterodimeric ZFNs containing FokI domains wherein the FokI domains comprise modified dimerization interfaces whereby only the heterodimeric FokI reconstituted species display catalytic activity (Szczepek et al., Structure-based redesign of the dimerization interface reduces the toxicity of zinc-finger nucleases, Nature Biotechnology, 2007). In certain embodiments, the Drp1 inhibitor is a restriction enzyme that specifically cleaves a genomic sequence (i.e. DNM1L sequence) encoding Drp1, or a Drp1 promoter sequence.

“Transcription-activator like effector nucleases”, or “TALENs” are artificial restriction enzymes that comprise a Transcription Activator Like (TAL) effector DNA binding domain which is fused to a DNA cleavage domain. In accordance with the rational engineering of zinc fingers, it is possible to engineer TAL effector domains to specifically bind any given DNA sequence present in a genome. Typical for TAL effector domains is that they comprise a repeated highly conserved 33 or 34 amino acid sequence with variable amino acids at the 12^{th} and 13^{th} position, commonly annotated as repeat variable diresidues, which are highly variable and show a strong correlation with specific nucleotide recognition patterns. Tools and protocols to generate TAL effector domains specific for a desired sequence are publicly available (Heigwer et al., E-TALEN: a web tool to design TALENs for genome engineering, Nucleic acids research, 2013, and Neff et al., Mojo Hand, a TALEN design tool for genome editing applications, BioMedCentral Bioinformatics, 2013). In accordance with the above-described ZFNs, TALENs may be based on the use a (modified) FokI domain as DNA cleavage domain, but can in theory include any DNA cleavage domain.

The term “CRISPR-associated (Cas)-based nucleases”, which may be used interchangeably with “CRISPR/Cas nuclease” is indicative for an enzyme, more specifically an endonuclease, that relies on the use of Clustered Regularly Interspaced Short Palindromic Repeats (CRISPR) sequences to recognize and cleave specific strands of nucleotide sequences that are complementary to the CRISPR sequence. CRISPR/Cas is a prokaryotic immune system conferring a prokaryotic defence mechanism to foreign nucleotide sequences. CRISPR/Cas systems are regarded in the state of the art as an acquired immune system in prokaryotes. Both single stranded, double stranded, RNA and DNA cleaving CRISPR/Cas systems have been described in the art and are therefore known to a person skilled in the art (Makarova and Koonin, Annotation and classification of CRISPR-Cas systems, Methods in molecular biology, 2015, and Makarova et al., Classification and nomenclature of CRISPR-Cas systems: where from here?, The CRISPR journal, 2018). By means of guidance and not limitation, examples of Cas proteins include Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, Cas9, Csn2, Cas4, C2c1, C2c3, Cas12 (i.e. Cpf1), Cas13a, Cas13b, Cas13c, and Cas13d. It is evident for a skilled person that different Cas proteins require different CRISPR sequences. Thus, in certain embodiments the CRISPR/Cas system specifically cleaves a genomic sequence encoding Drp1, or the Drp1 (i.e. DNM1L) promoter sequence.

In certain embodiments wherein the inhibitor of Drp1 is based on targeted activity of Cas9, the Cas9 protein is a modified Cas9 protein comprising a mutagenized HNH and/or RuvC catalytic domain. In further embodiments, the Cas9 protein is a nickase Cas9 (nCas9). In yet alternative embodiments, the Cas9 protein is a catalytically inactive Cas9 (i.e. dead Cas9, or dCas9). In such embodiments, double stranded DNA breaks may be achieved by fusing the catalytically inactive Cas9 protein to a DNA cleavage domain such as the non-limiting example FokI. Alternatively to the crRNA and tracrRNA, Cas9 may function by interaction with a single guide RNA (gRNA) sequence. Single guide RNA sequences have been designed artificially and are able to replace the crRNA and tracrRNA. It is known that the single guide RNA is a single RNA chimera of tracrRNA and crRNA. The crRNA or gRNA sequence comprises the sequence which the Cas9 will be targeted to by conventional base pairing of the crRNA or gRNA with the target sequence. Hence in certain embodiments described herein, the crRNA sequence or target specific portion of the gRNA sequence has a length of 15, 16, 17, 18, 19, 20, 21, 22, 23, 24, 25, 26, 27, 28, 29, or 30 nucleotides. In certain embodiments, the single guide RNA or crRNA comprises a portion of the nucleotide encoded Drp1 (i.e. DNM1L) sequence. In further embodiments, the single guide RNA or crRNA comprises a portion of a sequence contained in the first, second, or third exon sequence of DNM1L. In alternative embodiments, the single guide RNA or crRNA comprises a portion of the endogenous DNM1L promoter sequence. Tools for designing gRNA sequences have been reported in the art. Non-limiting examples of CRISPR-Cas design tools, optionally comprising an off-target analysis module include Breaking-Cas, Cas-OFFinder, CASTING, CCTop, CHOPCHOP, CHOPCHOP v2, CRISPOR, CRISPR Design, CRISPRdirect, CRISPRscan, CRISPRseek, DESKGEN, GuideScan, GT-Scan, Off-Spotter, sgRNA Designer, Synthego Design Tool, TUSCAN, and VARSCOT.

In alternative embodiments, the Drp1 inhibitor directly inhibits the function (i.e. activity) of Drp1. In certain embodiments, the Drp1 inhibitor decreases the activity of a Drp1 protein by binding to said Drp1 protein preferably by at least 2-fold, at least 4-fold, at least 6-fold, at least 8-fold, at least 10-fold when compared to the control activity of Drp1 without the addition of the inhibitor or compared to activity of Drp1 before the addition of the inhibitor. In certain embodiments, the inhibitor of Drp1 decreases the activity of a Drp1 protein by 100%. In these embodiments, it is understood that the Drp1 inhibitor achieved complete inhibition of Drp1 activity. Optionally, the Drp1 binding molecules are selected from the group consisting essentially of Drp1 binding antibodies, Drp1 antibody fragments or scaffolds, Drp1 binding proteins, Drp1 binding peptidomimetics, Drp1 binding aptamers, Drp1 binding photoaptamers, Drp1 binding spiegelmers, Drp1 binding nucleic acids and other Drp1-binding antibody mimetics such as Alphabodies, a Designed Ankyrin Repeat Proteins (DARPins), Monobodies, Affibodies, Anticalins, Avimers, Versabodies, and Uocalins and the like.

In certain embodiments, the Drp1 inhibitor is a Drp1 binding antibody. The term “antibody” as used herein is to be interpreted its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Furthermore, the term “antibody” is indicative for antibodies described herein, regardless of whether they are produced in vitro or in vivo. In certain embodiments, the Drp1 inhibitor is a Drp1 binding antibody that directly binds at least one functional domain, or an epitope comprised in the Drp1 protein.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method described in the art and known to a skilled person (Kohler et al., Continuous cultures of fused cells secreting antibody of predefined specificity., Nature, 1975). Alternatively, a skilled person is aware that antibodies can be made by recombinant DNA methods (Boss et al., Assembly of functional antibodies from immunoglobulin heavy and light chains synthesised in E. coli, Nucleic Acids Research, 1984). As a further non-limiting example, monoclonal antibodies can also be generated by relying on the use of phage display libraries (Clarckson et al., Making antibody fragments using phage display libraries, Nature 1991).

In certain embodiments, the Drp1 inhibitor is an antibody fragment. The term “antibody fragments” comprises a portion of an intact antibody, comprising the antigen-binding or variable region thereof. Methods to produce and purify antibody fragments are well established in the art (Bates and Power, David vs. Goliath: The Structure, Function, and Clinical Prospects of Antibody Fragments, Antibodies (Basel), 2019). By means of guidance and not limitation, examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning. In certain embodiments, the Drp1 inhibitor is an antibody fragment that directly binds at least one functional domain, or an epitope comprised in the Drp1 protein.

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

In certain embodiments, the agent may be a Nanobody. The terms “Nanobody” and “Nanobodies” are trademarks of Ablynx NV (Belgium). The term “Nanobody” is well-known in the art and as used herein in its broadest sense encompasses an immunological binding agent obtained (1) by isolating the VHH domain of a heavy-chain antibody, preferably a heavy-chain antibody derived from camelids; (2) by expression of a nucleotide sequence encoding a VHH domain; (3) by “humanization” of a naturally occurring VHH domain or by expression of a nucleic acid encoding a such humanized VHH domain; (4) by “camelization” of a VH domain from any animal species, and in particular from a mammalian species, such as from a human being, or by expression of a nucleic acid encoding such a camelized VH domain; (5) by “camelization” of a “domain antibody” or “dAb” as described in the art, or by expression of a nucleic acid encoding such a camelized dAb; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. “Camelids” as used herein comprise old world camelids (Camelus bactrianus and Camelus dromaderius) and new world camelids (for example Lama paccos, Lama glama and Lama vicugna). It is known to a person skilled in the art that, depending on the specific situation, nanobodies may display favourable characteristics compared to “traditional” antibodies, including but not limited to a high production yield in a broad variety of expression systems, minimal size, great stability, reversible refolding, and solubility in aqueous solutions.

In certain embodiments, the Drp1 inhibitor is an antibody-like scaffold or antibody mimetic. The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat). Methods and protocols to generate antibody-like protein scaffolds have been extensively reported in the art and are therefore known to a skilled person (inter alia in Skerra, Alternative non-antibody scaffolds for molecular recognition, Current opinion in biotechnology, 2007). Non-limiting examples of antibody-like protein scaffolds include affibodies, based on the Z-domain of staphylococcal protein A (Nygren, Alternative binding proteins: affibody binding proteins developed from a small three-helix bundle scaffold, Federation of European Biochemical Societies (FEBS) journal, 2008); engineered Kunitz domains based on a small (ca. 58 residues) and robust, disulphide-crosslinked serine protease inhibitor (Nixon and Wood, Engineered protein inhibitors of proteases, Current opinion in drug discovery & development, 2006); monobodies or adnectins based on the 10^{th} extracellular domain of human fibronectin III (10Fn3) that adopt an Ig-like beta-sandwich fold with 2 to 3 exposed loops, but lack the central disulphide bridge (Koide and Koide, Monobodies: antibody mimics based on the scaffold of the fibronectin type III domain, Methods in molecular biology, 2007); anticalins derived from the lipocalins, a diverse family of eight-stranded beta-barrel proteins that naturally form binding sites for small ligands by means of four structurally variable loops at the open end (Skerra, Alternative binding proteins: anticalins-harnessing the structural plasticity of the lipocalin ligand pocket to engineer novel binding activities, FEBS journal, 2008); DARPins, which are designed ankyrin repeat domains (Stumpp et al., DARPins: a new generation of protein therapeutics, Drug Discovery Today, 2008); avimers (Silverman et al., Multivalent avimer proteins evolved by exon shuffling of a family of human receptor domains, Nature Biotechnology, 2005); and cysteine-rich knottin peptides (Kolmar, Alternative binding proteins: Biological activity and therapeutic potential of cystine-knot miniproteins, FEBS journal, 2008).

In certain embodiments, the Drp1 inhibitor is an aptamer. The term “aptamer” as used herein refers to single-stranded or double-stranded oligo-DNA, oligo-RNA or oligo-DNA/RNA or any analogue thereof that specifically binds to a target molecule such as a peptide. Advantageously, aptamers display fairly high specificity and affinity (e.g., KA in the order 1×10⁹ M−1) for their targets. Methods for aptamer production and characterisation are described inter alia in U.S. Pat. No. 5,270,163 and Klussmann “The Aptamer Handbook: Functional Oligonucleotides and Their Applications”, Wiley-VCH 2006, ISBN 3527310592. The term “photoaptamer” refers to an aptamer that contains one or more photoreactive functional groups that can covalently bind to or crosslink with a target molecule. The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides. The term “peptidomimetic” refers to a non-peptide agent that is a topological analogue of a corresponding peptide. Methods of rationally designing peptidomimetics of peptides are known in the art (Perez, Designing peptidomimetics, Current topics in medicinal chemistry, 2018). Hence, in embodiments as described herein the Drp1 inhibitor is a Drp1 binding aptamer, i.e. a single-stranded or double-stranded oligo-DNA, oligo-RNA or oligo-DNA/RNA or any analogue thereof that specifically binds to Drp1.

By means of example and not limitation, the population of immune cells used for treatment with the Drp1 inhibitor may be obtained from peripheral blood (i.e. by means of a peripheral blood sample). The peripheral blood sample may comprise peripheral blood mononuclear cells (PBMCs) which include lymphocytes (e.g., T-cells, B-cells, NK cells, etc.) and monocytes. Optionally, the immune cells may be enriched or isolated from a peripheral blood sample. As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition. Enrichment methods and strategies for distinct cell types have been described at numerous instances throughout the art. Such enrichment strategies may result in a final cell population in which the percentage of one type of cell or subtype by at least about 1%, preferably by at least about 2%, preferably by at least about 5%, preferably by at least about 10%, preferably by at least about 20%, preferably by at least about 30%, preferably by at least about 40%, preferably by at least about 50%, more preferably by more than about 50%, more preferably by more than 60%, more preferably by more than about 70%, more preferably by more than about 80% when compared to the percentage of the one type of cell in a starting or initial population of cells. Similarly, “isolated” refers to a cell or a cell population that is removed from its natural environment (such as the peripheral blood) and that is isolated, purified or separated, and is at least about 50%, preferably at least about 60%, preferably at least about 70%, preferably at least about 75%, preferably at least about 80%, preferably at least about 85%, more preferably at least about 90%, more preferably about 95% or more free from other cell types with which it is naturally present. Optionally, the isolation is carried out by enrichment for a particular cell population by positive selection, or depletion of a particular cell population, by negative selection. In some embodiments, positive or negative selection is accomplished by incubating cells with one or more antigen-binding molecules or binding partners that specifically bind to one or more surface markers expressed or expressed at a relatively higher level on the respectively positively or negatively selected cells.

Optionally, the population of immune cells may be obtained by depleting a peripheral blood cell sample of one or more unwanted cell types. Thus, “depleting” when referring to one or more particular cell type or cell population, indicates decreasing the number or percentage of the cell type or population, when compared to the total number of cells in sample, or relative to other cell types, such as by negative selection based on markers expressed by the population or cell, or by positive selection based on a marker not present on the cell population or cell to be depleted.

Optionally, prior to and/or after contacting the population of immune cells with the Drp1 inhibitor, said population may be subjected to one or more differentiation steps or procedures. A skilled person readily appreciates that the term “differentiation” refers to a process of decreasing the potency or proliferation of a cell or moving the cell to a more developmentally restricted state.

Optionally, the population of immune cells may be expanded prior and/or after contacting said population of cells with the Drp1 inhibitor and thus represent an expanded population of immune cells. The term “expanded population” refers to a population of cells wherein a considerably portion of the cells (e.g. at least about 50%, preferably at least about 75%, preferably at least about 90%) of the cells have divided at least once after isolation from the peripheral blood and (an) optional enrichment steps. Consequently, the term “expanding” in the present context refers to an increase in cell number.

Preferred immune cells in the context of the present invention are natural killer (NK) cells or T cells.

Optionally, the (population of) immune cells described herein are (a population of) T cells, preferably antigen-specific T cells. As used herein, the term “antigen-specific T cells” refers to T cells that proliferate upon exposure to Antigen Presenting Cells (APCs) which present an antigen in the context of MHC and optionally at least one T cell co-stimulatory molecule. The term “antigen-specific T cells” also refers to T cells that are able to target (i.e. “attack”) cells having the specific antigen on their surfaces. Such T cells, for example cytotoxic T lymphocytes (CTLs), may eliminate target cells by different mechanisms. By means of example and not limitation, these mechanisms include the release of toxic enzymes such as granzymes and perforin to target cells. Generally, CTLs express CD8 on their cell surface. Alternatively, T cells expressing the CD4 antigen are routinely referred to in the art as “helper” T cells. Helper T cells are capable of promoting specific cytotoxic activity and may also be activated by APCs. Optionally, the T cells are selected from the group consisting of: CD4+ T helper (Th) cells (including Th1, Th2, Th17, and Tfh cells), CD8+ cytotoxic T lymphocytes (CTLs), regulatory T cells (Tregs), Natural Killer T cells (NKT cells), gamma delta T cells, memory T cells, mucosal-associated invariant T (MAIT) cells, invariant Natural Killer T (iNKT) cells, or any combination thereof.

Preferably, the (population of) immune cells described herein are (a population of) NK cells. NK cells are particularly preferred in the context of the present invention given to their particular biological that have been described at numerous instances throughout the art. NK cells are innate lymphocytes that recognize and clear infected and transformed cells without the need for prior sensitization. In addition, they exert an immunomodulatory function by excreting chemokines and cytokines, shaping the adaptive immune response. As used herein, the term “natural killer cells” or “NK cells” refers to a type of lymphocytes involved in the innate immune response. NK cells exhibit cytolytic activity against a variety of targets via exocytosis of cytoplasmic granules containing a variety of proteins, including perforin, granulysin and granzyme proteases. NK cells occur in both lymphoid and nonlymphoid tissues and are readily distinguishable from lymphocytes of the adaptive immune system (e.g. T cells and B cells). In general, NK cells are further characterized by the absence of CD3 or antigen-specific receptors at their cell surface. In humans, CD56 is a commonly used marker for NK cells. NK cells are involved in rapid response to detect and clear virus-infected cells and immune surveillance of tumor cells without the requirement of prior immunization. Optionally, the NK cells are selected from the group consisting of: CD56bright NK cells, CD56dim NK cells, adaptive NK cells, CD16+ NK cells, CD16− NK cells, liver-resident NK cells, decidual NK cells, or any combination thereof.

Optionally, the NK cells are primary NK cells purified from peripheral blood of a subject. The purification method is not particularly limiting in the context of the invention. By means of illustration and not limitation, the NK cells may be purified by for example leukapheresis or density gradient centrifugation.

Alternatively, the NK cells may be immortalized NK cells. It is understood that “immortalized cells” in the present context indicates cells that have acquired an infinite proliferation ability and are not, or to a negligible extent affected by cell senescence. The use of immortalized natural killer cells is of particular interest in the present context to generate an off-the shelf product for cell therapy. In particular embodiments, the immortal natural killer cells are NK-92 cells or NK-92 MI cells. Both cell lines are commercially available, for example from the American Type Culture Collection (ATCC) under the respective identifiers CRL-2407 and CRL-2408. Alternative cell lines similar to NK-92 cells are known in the art, such as the NK101 cell line (Yang et al. Journal for ImmunoTherapy of Cancer (2019) 7:138)

Yet alternative sources of NK cells known to a person skilled in the art include cord blood and differentiation from induced pluripotent stem cells (iPSCs).

Particularly envisaged in the context of the present invention is the reduction or inhibition of the Drp1 expression level and/or Drp1 activity in immune cells (i.e., obtaining hypoxia resistance in immune cells) that comprise modifications vis-à-vis naturally occurring immune cells that aim to achieve enhanced antigen recognition and/or enhanced potency. Such manipulated immune cells are termed in different manners in the art, such as “armed immune cells”, “augmented immune cells”, “redirected immune cells”, “immune cells containing specific homing abilities”, “weaponised immune cells”, and the like. Hence, in each instance wherein reference is made to immune cells a skilled person readily appreciates that this expression also includes and envisages redirected immune cells.

The term “redirected immune cells” should be interpreted in its broadest manner as found throughout the art and therefore indicates immune cells that have been manipulated in any way to improve potency towards cells expressing a particular antigen or a group (i.e., collection) of antigens. The expression “redirected” is therefore not to be interpreted as limited to genetic manipulation and also includes redirecting by interaction of the immune cells with moieties that enable or improve binding to one or more particular antigen or a group of antigens.

Hence, optionally the (redirected) immune cells are cells that are bound to a mono-, bi- and/or multispecific affinity ligand. A person of ordinary skill in the art appreciates that the term “multispecific affinity ligands” refers to molecules designed to simultaneously bind to at least two different targets. Optionally, the multispecific affinity ligand may be expressed, secreted, and/or displayed by the immune cells. In alternative further embodiments, the multispecific affinity ligand is introduced to the (population of) immune cell(s) by further manipulation. In such embodiments, the redirected immune cells themselves do not express the multispecific affinity ligand. In typical embodiments, said multispecific affinity ligands bind both to the (surface of) the immune cell and an antigen, which may optionally be a tumour antigen. The multispecific affinity ligand may be introduced to the (population of) immune cells prior to, concomitant with, or after the one or more manipulations that reduce or inhibit the Drp1 expression level and/or activity. The present disclosure envisages all kinds or multispecific affinity ligands and therefore include by means of illustration but not limitation multispecific or bispecific (monoclonal) antibodies (BsAbs or BsMAbs), multispecific or bispecific T cell engagers (BiTEs) and NK cell engagers, dual targeting peptides, bispecific aptamers, bispecific small molecules, bispecific fusion proteins or peptides, multispecific or bispecific affibodies, and multispecific or bispecific nucleic acid aptamers. It is to be appreciated that these redirected immune cells have been manipulated to achieve “engaging therapy”. “Engaging therapy” as used herein refers to a plethora of approaches aiming to (re-)direct said immune cells towards the tumour, in an antigen-specific or non-specific manner, and/or to activate said immune cells, in an external (via a molecule) or an internal (engineering) approach. Summarizing illustrative examples to achieve this include without limitation (i) antibodies, including those that can potentially elicit antibody-dependent cellular cytotoxicity, (ii) immune cell engagers, including mono-, bi-, tri-, tetra-, pentaspecific engagers, (iii) chimeric antigens receptors, or CARs, (iv) T-cell receptors, or TCRs, (v) high-affinity receptors, including haCD16, (vi) protease resistant FcR, (vii) dominant-negative receptors, (viii) chemokine receptors, (ix) cytokine receptors, (x) nanomolecules, or any combination thereof.

Hence optionally, in addition to having a reduced or inhibited Drp1 expression level and/or activity, the immune cells are bound to (and optionally express, display, and/or secrete) a multispecific or bispecific affinity ligand. Optionally, the immune cells are bound to (and optionally express, display, and/or secrete) a multispecific or bispecific antibody.

A typical example of monospecific affinity ligands such as monospecific antibodies are antibodies capable of eliciting Antibody-Dependent Cellular Cytotoxicity (ADCC). A skilled person is aware of the concept ADCC and how such antibodies can complement the cells subject of the present disclosure.

Optionally and alternatively or in addition to the above embodiments describing the occurrence of mono-, bi- or multispecific affinity ligands, in addition to having a reduced or inhibited Drp1 expression level and/or activity, the immune cells express a chimeric antigen receptor (CAR) and/or an artificial (i.e. heterologous) T cell receptor (TCR).

The term “chimeric antigen receptor” and the corresponding abbreviation “CAR” refer to an artificially constructed hybrid polypeptide having a basic structure that includes an antigen-binding region, a transmembrane domain, a co-stimulatory domain, and an intracellular signalling domain. A CAR can redirect the specificity and reactivity of immune cells (such as for example T cells and NK cells) to a defined target by utilizing the antigen binding properties of monoclonal antibodies. As used herein, “antigen-binding region” refers to any structure or a functional variant thereof that can bind to an antigen. The antigen-binding region may be an antibody structure, including, but not limited to, monoclonal antibody, polyclonal antibody, recombinant antibody, human antibody, humanized antibody, chimeric antibody, and functional fragment thereof. For example, the different kinds of antigen-binding regions include those described above as possible entities that reduce Drp1. Thus, by means of example but not limitation such antigen-binding regions include but are not limited to, Fabs, single chain antibody fragments (scFvs), single domain antibodies (sdAbs), nanobodies (Nbs), antigen binding ligands, recombinant fibronectin domains, anticalins, and DARPINs. The antigen-binding region may be monovalent or bivalent, and may be a monospecific, bispecific or multispecific antibody. In another embodiment, the antigen-binding region also may be a specific binding polypeptide or receptor of a specific protein, wherein the specific protein is, for example, Muc-4, Mesothelin, CD70, or Fap. Further examples of specific proteins that may be targeted by the CAR include by means of illustration and not limitation: AFP, ALPP, AXL, B7-H3, BAFFR, BCMA, C182A, C7R, CAIX, CD10, CD117, CD123, CD124, CD126, CD13, CD133, CD138, CD147, CD171, CD19, CD20, CD22, CD269, CD276, CD3, CD30, CD33, CD34, CD37, CD38, CD4, CD44v6, CD5, CD56, CD7, CD70, CD73, CD79b, CD80, CEA, Chlorotoxin, CLDN 18.2, CLDN6, CLL-1, c-Met, CS1, DLL3, DR5, DSG3, EGFR, EPCAM, EphA2, ErbB, FAP, FLT3, FRa, GD2, GFRa4, GLY, GP100, GP120, gp-120, GPC3, GPRC5D, GUCY2C, Her2, HLA-A2, ICAM-1, IL13Ra2, ILIRAP, ILT3, Integrin $7, Kappa-light chain, KLK2, LCAR-T2C, LeY, LMP1, MG7, MSLN, MUC1, MUC16, Nectin4, NKG2D, NKG2DL, NKR-2, NY-ESO-1, PD-1, PD-L1, PII3K, PSCA, PSMA, ROR1, ROR2, SENL-T7, SLAMF7, TM4SF1-, TnMUC1, TRBC1, TSLPR, and VEGFR2.

Approaches to construct CARs have been described in numerous instances throughout the art and are therefore known to a skilled person (see for example Guedan et al., Engineering and Design of Chimeric Antigen Receptors, Mol Ther Methods Clin Dev, 2018). A further illustrative example of a CAR is CD70-CAR-IL-15, which can suitably be introduced into the immune cells described herein by means of electroporation of CD70-CAR-IL-15 encoding mRNA, such as but not limited to nucleofection of CD70-CAR-IL-15 encoding mRNA.

The term “T cell receptor” and the corresponding abbreviation “TCR” refers to a protein typically occurring on the surface of T cells. TCRs are crucial for recognizing and interacting with specific antigens. In the context of the present invention the hypoxia-resistant immune cells may express one or more heterologous or artificial (i.e. modified and/or genetically engineered) TCRs on their surface.

Optionally, in addition to having a reduced or inhibited Drp1 expression level and/or activity, the immune cells are further modified for survival and/or efficacy in a solid tumour microenvironment.

For example, the immune cells may be modified to have reduced mitochondrial Reactive Oxygen Species (ROS) expression. Numerous strategies for manipulating ROS have been described in the art and are therefore known to a skilled person. By means of example and not limitation, such strategies include the use of antioxidants such as mitochondrial-targeted antioxidants, the use of ROS scavengers, inhibition of NADPH oxidase, and/or inhibition of the mTOR pathway.

An exemplary antioxidant is, N-acetylcysteine (NAC), a precursor of glutathione, which may increase the antioxidant capacity of immune cells and reduce ROS levels. An exemplary ROS scavenger is the enzyme catalase. Catalase has the capacity to convert hydrogen peroxide to water and oxygen, reducing the levels of ROS. Exemplary mitochondrial-targeted antioxidants include MitoQ and MitoTEMPO, which can selectively accumulate in mitochondria and reduce ROS levels. An exemplary NADPH oxidase inhibitor is diphenylene iodonium (DPI) which can inhibit NADPH oxidase activity in T cells and enhance their anti-cancer activity. An exemplary MTOR inhibitor is rapamycin. Rapamycin is known to reduce ROS levels in T cells and enhance their anti-cancer activity.

In yet a further possible or alternative embodiment, the uses and treatments described herein may combine the use of (optionally redirected) hypoxia-resistant immune cells characterised by a reduced Drp1 expression and/or activity with further means that have been described in the art to treat cancers such as solid tumours and leukaemia.

Hence, optionally oxygen-carrying nanoparticles may be used to complement the uses and treatments described herein. For instance, oxygen-carrying nanoparticles have been shown to enhance the efficacy of adoptive cell therapy in a hypoxic tumour environment (Li et al, Front Mol Biosci, 2021). Such nanoparticles may carry oxygen and release it in hypoxic conditions, which can enhance the survival and function of immune cells.

Alternatively, modulation of checkpoint inhibitors may assist the hypoxia resistant immune cells described herein to treat a cancer such as a solid tumour or leukaemia. By means of illustration and not limitation, inhibition of the programmed death-1 (PD-1) checkpoint can enhance the function of T cells in a hypoxic tumour environment (Hu et al., PeerJ, 2021). The solid tumour cancer may be, or may considered to be a solid tumour comprising a microenvironment characterised by a local oxygen level of less than about 8%, preferably of less than about 7%, preferably of less than about 6%, preferably of less than about 5%, more preferably of less than about 4%, more preferably of less than about 3%, more preferably of less than about 2%, most preferably of less than about 1%. Hence, the hypoxia-resistant immune cells characterised by a reduced Drp1 expression level may be used to treat a tumour microenvironment having local oxygen levels of from about 0% to about 8%, preferably of from about 0.5% to about 7%, preferably of from about 1% to about 6%, more preferably of from about 1.5% to about 5%.

Preferably, the Drp1 expression in the population of hypoxia-resistant immune cells is reduced by means of a chemical inhibitor for a period of at least about 24 hours prior to introduction into (i.e. administration to) a subject by contacting immune cells with a Drp1 inhibitor to generate the hypoxia-resistant immune cells. Preferably, the Drp1 expression in the population of hypoxia-resistant immune cells is reduced by means of a chemical inhibitor for a period of at least about 36 hours, preferably at least about 48 hours, preferably at least about 60 hours, more preferably at least about 72 hours, prior to introduction into (i.e. administration to) a subject by contacting immune cells with a Drp1 inhibitor to generate the hypoxia-resistant immune cells. The hypoxia-resistant immune cells used in the context of the present disclosure are therefore preferably characterised by prolonged reduced Drp1 expression. In certain embodiments, the hypoxia-resistant immune cells are used that prior to administration have experienced a reduced Drp1 expression level for at least about 24 hours, preferably at least about 36 hours, preferably at least about 48 hours, preferably at least about 60 hours, more preferably at least about 72 hours.

In embodiments wherein the Drp1 activity in the population of hypoxia-resistant immune cells is reduced by means of a chemical inhibitor such as Mdivi-1, the Drp1 activity is reduced by contacting the cells with at least about 1 μM, preferably at least about 2.5 μM, more preferably at least about 5 μM, most preferably at least about 10 μM of the chemical inhibitor such as Mdivi-1.

In further preferred embodiments, the Drp1 activity in the population of hypoxia-resistant immune cells is reduced by means of contacting said cells with at least about 10 μM Mdivi-1 for at least about 72 hours. The inventors have observed that this duration and concentration is particularly beneficial to produce hypoxia-resistant immune cells that have improved properties such as those described throughout the present disclosure when compared to hypoxia-resistant immune cells described in the art.

Optionally, the immune cells comprise a kill switch mechanism. “Kill switch mechanism” as described herein refer to artificially introduced cellular mechanisms (and thus cellular components responsible for exerting the mechanism) that provide a safety feature to gene therapy and cell-based therapies by controlling the survival or death of engineered cells in response to specific stimuli. A skilled person appreciates that a plethora of kill switch mechanisms have been described throughout the art and understands that the choice for a particular kill switch mechanism depends on the specific application and the desired outcomes. Illustrative and non-limiting kill switch mechanisms that are envisaged for the hypoxia-resistant immune cell include inducible Caspase-9 (iCasp9) systems, synthetic Notch (synNotch) receptor systems, Herpes Simplex Virus Thymidine Kinase (HSV-TK) systems, and riboswitch systems.

The iCasp9 system is based on the use of a modified Caspase-9 enzyme that can be activated by a small molecule (e.g. AP1903) (Gargett and Brown, Front Pharmacol, 2014). The iCasp9 gene is typically introduced into the target cells, in the present context the immune cells that may have been rendered hypoxia-resistant or will be rendered hypoxia-resistant in the foreseeable future (in the present context the hypoxia-resistance is achieved by reducing Drp1 expression). In the absence of the small molecule, the iCasp9 enzyme remains inactive. Upon addition of AP1903, the iCasp9 enzyme becomes activated, triggering a cascade of events which ultimately result in apoptosis of the target cells.

The synNotch receptor system is an alternative kill switch mechanism wherein a synNotch receptor (i.e. a synthetic receptor) is linked to a transcriptional activator that drives the expression of a suicide gene or a pro-apoptotic gene in response to ligand binding (Choe et al., Sci Transl Med, 2021).

Further alternative kill switch mechanisms are based on HSV-TK systems, which is directed to the use of the herpes simplex virus thymidine kinase enzyme. The HSV-TK enzyme is inactive in absence of ganciclovir. Upon addition of ganciclovir, the HSV-TK enzyme becomes active, leading to the phosphorylation of ganciclovir and ultimately, the death of the target cells (Greco et al., Front Pharmacol, 2015).

Further illustrative kill switch systems may be based on riboswitch systems. A riboswitch kill switch system is generally based on the use of RNA molecules that can bind to specific ligands and undergo conformational changes that lead to the activation of a downstream gene. The riboswitch can be engineered to recognize specific ligands and activate the expression of a suicide gene or a pro-apoptotic gene in response to ligand binding (Sullenger, N Engl J Med, 2004).

Optionally, the (optionally redirected) hypoxia-resistant immune cells are adapted to be auxotrophic. The term “auxotrophic” refers to the inability of a cell to synthesize a particular nutrient or compound that is essential for proliferation (i.e. cell division and/or survival). Thus, an auxotrophic cell requires a specific nutrient or compound that cannot be synthesized endogenously but must be supplied exogenously.

Auxotrophic cells are often used in molecular biology and genetic engineering to study cellular metabolism, gene regulation, and gene expression. They are also used in biotechnology to produce specific compounds or recombinant proteins that require the exogenous supply of certain nutrients. By means of example and not limitation, auxotrophic versions of the hypoxia-resistant immune cells described herein may be generated by modification of one or more amino acid synthesis pathways, nucleotide synthesis pathways, vitamin synthesis pathways, coenzyme synthesis pathways, modification of the glucose metabolism, fatty acid synthesis pathways, or any combination thereof.

In certain embodiments, the Drp1 expression and/or activity is reduced in the population of hypoxia-resistant immune cells by at least at least about 75%, preferably by at least about 85%, more preferably by at least about 95%, most preferably by about 100% when compared to a non-hypoxia-resistant immune cell. It is understood that when reference is made to a reduction of about 100%, the residual Drp1 expression level and/or activity is at least below the detection limit of any method described in the art to evaluate respectively gene expression and/or protein functionality.

It is evident that the hypoxia-resistant immune cells described throughout the present disclosure are equally envisaged to be a component of a pharmaceutical composition. The hypoxia-resistant immune described comprised in the pharmaceutical composition may therefore be characterized by any of the herein described embodiments for the hypoxia-resistant immune cells as such, i.e. any embodiment described herein for the hypoxia-resistant cells is equally envisaged and optionally preferred for the hypoxia-resistant cells when part of a pharmaceutical composition. The terms “pharmaceutical composition”, “pharmaceutical formulation”, or “pharmaceutical preparation” may be used interchangeably herein and are intended to describe compositions and formulations containing a population of hypoxia-resistant immune cells described herein as active pharmaceutical ingredient, optionally formulated with a pharmaceutically acceptable excipient, and manufactured or sold with the approval of a governmental regulatory agency as part of a therapeutic regimen for the treatment of disease in a mammal, such as cancer in a human subject. “Active pharmaceutical ingredient” or “API” as referred to herein is to be interpreted according to the definition of the term by the World Health organization: a substance used in a finished pharmaceutical product (FPP), intended to display pharmacological activity or to otherwise have direct effect in the diagnosis, cure, mitigation, treatment or prevention of disease, or to have direct effect in restoring, correcting or modifying physiological functions in human beings. It is evident that pharmaceutical compositions in the context of the invention are indicative for those compositions that comprise a therapeutically or effective amount of the hypoxia-resistant immune cells that are characterised by a reduced Drp1 expression and/or activity.

It is evident and envisaged that the pharmaceutical compositions described herein may comprise one or more suitable excipients. Techniques regarding the formulation and administration of pharmaceutical compositions are known to a skilled person and have been described in the art (e.g. the reference book: Remington: The Science and Practice of Pharmacy, periodically revised). The term “excipient” as used interchangeably herein and in the art with “carrier” may be indicative for any solvent, diluent, buffer (including but not limited to neutral buffered saline, phosphate buffered saline, or optionally Tris-HCl, acetate or phosphate buffers), solubiliser (including but not limited to Tween 80 or Polysorbate 80), colloid, dispersion medium, vehicle, filler, chelating agent (including but not limited to EDTA or glutathione), amino acid, protein, disintegrant, binder, lubricant, wetting agent, stabiliser, emulsifier, sweetener, colorant, flavouring, aromatiser, thickener, any agent suitable to achieve a depot effect, coating, antifungal agent, any preservative (including but not limited to Thimerosal™, benzalkonium chloride, or benzyl alcohol), antioxidant (including but not limited to ascorbic acid, sodium metabisulfite), tonicity controlling agent, absorption delaying agent, adjuvant, bulking agent (including but not limited to lactose, mannitol) and any other ingredient that may influence any parameter or characteristic of the composition subject of the invention. A skilled person understands that one or more excipients may be used in the composition on condition that the one or more excipient is compatible with the one or more pharmaceutical ingredient (i.e. in the context of the present invention at least the hypoxia-resistant immune cells) and that a pharmaceutically acceptable formulation is obtained.

In certain embodiments, the excipient may be an active pharmaceutical ingredient excipient, binder excipient, carrier excipient, co-processed excipient, coating system excipient, controlled release excipient, diluent excipient, disintegrant excipient, dry powder inhalation excipient, effervescent system excipient, emulsifier excipient, lipid excipient, lubricant excipient, modified release excipient, penetration enhancer excipient, permeation enhancer excipient, pH modifier excipient, plasticiser excipient, preservative excipient, preservative excipient, solubiliser excipient, solvent excipient, sustained release excipient, sweetener excipient, taste making excipient, thickener excipient, viscosity modifier excipient, filler excipient, compaction excipient, dry granulation excipient, hot melt extrusion excipient, wet granulation excipient, rapid release agent excipient, increased bioavailability excipient, dispersion excipient, solubility enhancement excipient, stabilizer excipient, capsule filling excipient, or any combination hereof. A skilled person is aware that use of such media and agents for pharmaceutical active substances is common practice and incorporation of these excipients is hence well known in the art. It is evident that all of the used ingredients should be non-toxic in the concentration contained in the final pharmaceutical composition and should not negatively interfere with the activity of the one or more pharmaceutically active ingredients, in this context at least the hypoxia-resistant immune cells.

Optionally, the hypoxia-resistant immune cells that are characterised by reduced Drp1 expression and/or activity are present in a pharmaceutical composition in a lyophilised form. In such embodiments, the immune cells are lyophilised. The terms “lyophilised” or “freeze-dried” can be used interchangeably herein and refer to a condition and/or state of a sample, formulation, or product obtained by means of lyophilisation. Lyophilisation, also known as freeze-drying or cryodesiccation, is a dehydration process which involves freezing the product without destroying the physical structure of the matter. Lyophilisation comprises at least a freezing step and a sublimation step. The sublimation step may comprise two stages of drying: a primary drying step and a secondary drying step. Lyophilisation may be used in the manufacturing of pharmaceutical products and intermediates thereof. During freezing, the material is cooled to a temperature wherein the solid, liquid, and gas phases of the material may exist. Advantages of lyophilisation may be but are not limited to improved aseptic handling, enhanced stability of a dry powder, the removal of water without excessive heating of the product, and enhanced product stability in a dry state. In general, the quality of a rehydrated, lyophilized product is excellent and does not show inferior (therapeutic) characteristics to a non-lyophilized product.

The pharmaceutical composition may hence contain the hypoxia-resistant immune cells in such a form that reconstitution is required prior to the medical use (i.e. administration to a subject). As used herein, “reconstitution” refers to the process of restoring a dried, lyophilized, dehydrated, or concentrated matter to its original or liquid state by adding a solvent to the lyophilized matter, preferably followed by agitating the mixture of the solvent and lyophilized matter. By means of example and not limitation, an aqueous solution or fluid may need to be added to the pharmaceutical composition. “Water for injection”, interchangeably indicated in the art by terms such as “aqua ad injectabilia” and “aqua ad injectionem” refers to water without any significant contamination suitable for injection to a person. As defined herein, the water is considered sterile and/or other substances are added to make the solution about isotonic. In certain embodiments, the aqueous solution may be a physiological saline or isotonic saline solution.

A further aspect of the invention is directed to a method of producing hypoxia-resistant immune cells having reduced or inhibited Drp1 expression and/or activity. The method comprises at least a step of treating immune cells with a specific Drp1 inhibitor. Preferably, the method comprises the steps of providing immune cells and treating said immune cells ex vivo with a specific Drp1 inhibitor. It is evident that any of the immune cells described herein are envisaged in the method of producing the hypoxia-resistant immune cells, as well any Drp1 inhibitors. Hence, optionally the immune cells may be derived from a subject as defined herein. The specific Drp1 inhibitor may therefore be any Drp1 inhibitor disclosed herein. Such Drp1 inhibitor may therefore be a Drp1 inhibitor selected from the group consisting of: chemical inhibitors, binding proteins, gene editing systems, or antisense agents as described herein. Particularly preferred Drp1 inhibitors are gene editing systems, such as gene editing systems, preferably CRISPR-Cas gene editing systems such as the CRISPR/Cas9 gene editing system. The origin of the immune cells is not particularly limiting, and may hence be directly derived from a subject, optionally the subject that is to be treated with the hypoxia-resistant immune cells, or immortalised immune cells.

In embodiments wherein the immune cells are derived (i.e. extracted) from a subject, optionally the subject that is to be treated with the hypoxia-resistant immune cells, the method may involve any means known in the art such as density gradient centrifugation, magnetic-activated cell sorting (MACS), fluorescence-activated cell sorting (FACS), and/or cell scraping.

Alternatively, immortalised cells not derived from the subject to be treated are used to generate the hypoxia-resistant immune cells having reduced or inhibited Drp1 expression and/or activity. Such cells are particularly preferred in the context of the present invention, since they allow for the provision of “off-the-shelf” cell products suitable for treating cancer. The expression “off-the-shelf” typically refers to a product or service that is readily available for purchase, without requiring any customization or special ordering. It suggests that the item is in stock and can be purchased immediately, without the need for any additional preparation or production time.

Optionally, the provided immune cells are cultured for a certain amount of time prior to contacting said cells with the Drp1 inhibitor. Cell culture parameters have been described at numerous instances in the art and are therefore known to a skilled person. By means of illustration, parameters that are to be considered upon culturing mammalian cells such as immune cells include cell density, material of the culture container (vessel), culture medium, gas exchange, and temperature.

Optionally, the method of producing hypoxia-resistant immune cells comprises a further step of enriching immune cells that have been successfully treated with the Drp1 inhibitor. In certain embodiments, a functional assay is conducted that allows for enrichment of immune cells treated with Drp1 inhibitor. Additionally and/or alternatively, a genetic assay is conducted that allows for enrichment of immune cells treated with Drp1 inhibitor. In further optional embodiments, a step of clonal selection occurs after treatment of the immune cells with Drp1 inhibitor. “Clonal selection” as used herein refers to a process in which a single cell is selected and amplified to produce a population of genetically identical cells, all derived from a single parent cell. The use of clonal selection has a number of advantages in the context of manipulating cells for research or therapeutic purposes, as known to a person skilled in the art. It allows for the generation of a homogeneous population of cells with a defined genetic profile and desirable characteristics, such as high expression of a particular protein or resistance to a specific drug. Hence, in such embodiments, a single immune cell wherein Drp1 expression and/or activity has been reduced or inhibited to a satisfactorily extend is selected and further amplified (i.e. cultured) to produce a population of genetically identical hypoxia-resistant immune cells before administration to a subject.

In preferred embodiments, the immune cells are treated with a Drp1 inhibitor that is a chemical inhibitor. Preferably, the Drp1 expression and/or activity in the provided immune cells is reduced by means of a chemical inhibitor for a period of at least about 24 hours to generate the hypoxia-resistant immune cells. Preferably, the Drp1 expression and/or activity in the provided immune cells is reduced by means of a chemical inhibitor for a period of at least about 36 hours, preferably at least about 48 hours, preferably at least about 60 hours, more preferably at least about 72 hours to generate the hypoxia-resistant immune cells. The method disclosed herein is thus preferably characterised by a prolonged step of treating immune cells with a specific Drp1 inhibitor. The inventors have observed that prolonged reduction or inhibition of Drp1 results in cells having favourable hypoxia-resistance in terms of viability, and consequently an improved efficacy upon introduction into a subject.

In embodiments wherein the Drp1 activity in the population of hypoxia-resistant immune cells is reduced by means of a chemical inhibitor such as Mdivi-1, the Drp1 activity is reduced by contacting the cells with at least about 1 μM, preferably at least about 2.5 μM, more preferably at least about 5 μM, most preferably at least about 10 μM of the chemical inhibitor such as Mdivi-1. Optionally, the Drp1 activity is reduced by contacting the cells with from about 0.2 μM to about 50 μm Midivi-1, preferably from about 0.5 μm to about 25 μm Mdivi-1, preferably from about 1 μM to about 20 μM Mdivi-1, more preferably from about 2 μM to about 15 μM Mdivi-1, more preferably from about 5 μM to about 12.5 μM Mdivi-1, most preferably about 10 μM Mdivi-1.

In further preferred embodiments, the Drp1 activity in the population of hypoxia-resistant immune cells is reduced by means of contacting said cells with at least about 10 μM Mdivi-1 for at least about 72 hours. The inventors have observed that this duration and concentration is particularly beneficial to produce hypoxia-resistant immune cells that have improved properties such as those described throughout the present disclosure when compared to hypoxia-resistant immune cells described in the art.

Optionally, treatment of the immune cells with the Drp1 inhibitor results in a reduction, in the cells of the present invention, of Drp1 expression and/or activity of at least about 50%, preferably at least about 65%, preferably at least about 75%, preferably at least about 85%, more preferably at least about 95%, most preferably by about 100% in the provided immune cells. Preferably, treatment of the immune cells with Drp1 inhibitor results in a population of immune cells (i.e. a population of hypoxia-resistant immune cells) that is characterised by an on population-level average Drp1 expression level and/or activity reduction of at least about 25%, more preferably at least about 50%, more preferably at least preferably at least about 65%, preferably at least about 75%, preferably at least about 85%, more preferably at least about 95%, most preferably by about 100%. In certain embodiments, treatment of the immune cells with the Drp1 inhibitor results in a population of immune cells (i.e. a population of hypoxia-resistant immune cells) wherein at least about 30%, preferably at least about 50%, preferably at least about 65%, preferably at least about 75%, preferably at least about 85%, more preferably at least about 95% of the cells that have been treated with the Drp1 inhibitor have a reduced Drp1 expression level and/or reduced Drp1 activity level, preferably an about 50% reduced Drp1 expression level and/or reduced Drp1 activity level, more preferably an about 75% reduced Drp1 expression level and/or reduced Drp1 activity level, more preferably an about 85% reduced Drp1 expression level and/or reduced Drp1 activity level, more preferably an about 95% reduced Drp1 expression level and/or reduced Drp1 activity level, most preferably about 100% reduced Drp1 expression level and/or reduced Drp1 activity level.

In certain embodiments, the Drp1 (expression and/or activity) reduction is transient and returns to a baseline expression level after about 1 hour post treatment with the Drp1 inhibitor, preferably after about 2 hours post treatment with the Drp1 inhibitor, preferably after about 4 hours post treatment with the Drp1 inhibitor, more preferably after about 6 hours post treatment with the Drp1 inhibitor, more preferably after about 12 hours post treatment with the Drp1 inhibitor, more preferably after about 24 hours post treatment with the Drp1 inhibitor, more preferably after about 48 hours post treatment with the Drp1 inhibitor, more preferably after about 72 hours post treatment with the Drp1 inhibitor.

In preferred embodiments, the Drp1 expression reduction and/or activity reduction is irreversible (i.e. permanent).

Optionally, in addition to contacting the immune cells with a DRP1 inhibitor (which may be for example a chemical compound or a gene editing system), the immune cells may be additionally contacted with a further specific modulator (which may be an inhibitor or an activator) of one or more proteins expressed in the immune cells. The further specific modulator may be any type of modulator such as the types of Drp1 inhibitors described herein. Thus, the immune cells may be treated with two or more chemical compounds (inhibitor or modulator), two or more gene editing systems, or any combination of at least one chemical compound and at least one gene editing system. In preferred embodiments, the gene editing system may be identical for Drp1 and the one or more other proteins with the exception of the specific targeting portion of the gene editing system (by means of illustration and not limitation a CRISPR/Cas system wherein at least two unrelated gRNA molecules are co-introduced into the cells).

In certain embodiments of the method, the cells are treated with a further specific inhibitor for reducing expression of a further protein. In alternative embodiments of the method, the cells are treated with a further specific activating agent for enhancing expression of a further protein. In yet alternative embodiments of the method, the cells are treated with an inhibitor for reducing expression of a further protein and treated with an inhibitor for reducing expression of a yet further protein. Exemplary proteins include but are not limited to those described herein.

In further embodiments, the method comprises a further step of introducing one or more heterologous genes into the genomic sequence of the immune cells. A preferred heterologous gene in the present context is a chimeric antigen receptor.

The invention thus provides in hypoxia-resistant immune cells that are produced by any embodiment of the methods described herein.

The particular method that is used to administer the population of cells or the pharmaceutical composition to a subject is not particularly limiting for the invention. Optionally, the administration method is selected from the group consisting of: intravenous (IV) infusion, intratumoral injection, intraperitoneal injection, intra-arterial infusion, intracavitary injection, inhalation, topical application, and oral administration. When using intravenous infusion, the immune cells are administered directly into the bloodstream through a catheter. When using intratumoral injection, the immune cells are directly injected into the tumour mass. When using intraperitoneal injection, the immune cells are injected into the peritoneal cavity, which is the space that surrounds the abdominal organs. When using intra-arterial infusion, the immune cells are infused directly into the artery that supplies blood to the tumour. When using intracavitary injection, the immune cells are injected into body cavities, such as the pleural or pericardial cavity. While other administration methods of immune cells to a subject such as inhalation, topical administration and oral administration is less widespread, said administration method may nonetheless be of particular interest to treat certain cancer (i.e. tumour) types.

The administration may be continuous. “Continuous” as used herein indicates that the population of hypoxia-resistant immune cells are administered at relatively regular intervals, with no (therapeutically) significant interruptions. Naturally, minor interruptions may occur that do not affect the overall effectiveness of the treatment, and indeed such aberrations are encompassed by the present invention. In a preferred embodiment, and more arithmetically, the administration regimen is deemed to be continuous if the longest interval between two subsequent administrations is not more than 3.5 times as long as the average interval. Even more preferably said longest interval is not more than 2.5 times, most preferably not more than 1.5 times as long as the average interval.

Optionally, the population of hypoxia-resistant immune cells are used in combination with known therapies known to be effective against cancer such as chemotherapy, immunotherapy, radiotherapy, surgery, vaccination, and/or hormone therapy. It is appreciated that the combination of the use of hypoxia-resistant immune cells and one or more of these known therapies may be indicated as a combination therapy. The known therapy or therapies may occur before, during and/or after the therapies based on the population of hypoxia-resistant immune cells described herein, and delivery thereof may occur via the same or different administration route(s) as the administration method used to administer the population of hypoxia-resistant immune cells subject of the invention.

Hence, by means of illustration the population of hypoxia-resistant cells may be used to treat a subject for cancer, wherein the subject is additionally treated by a molecule selected from the group consisting of: alkylating agents, antimetabolites, topoisomerase inhibitors, mitotic inhibitors, hormonal receptor targeting agents, monoclonal antibodies, immunomodulators, proteasome inhibitors, kinase inhibitors, DNA repair inhibitors. Illustrative examples of alkylating agents include cyclophosphamide, ifosfamide, and chlorambucil. Illustrative examples of antimetabolites include methotrexate, 5-fluorouracil, and gemcitabine. Illustrative examples of topoisomerase inhibitors include etoposide, doxorubicin, and irinotecan. Illustrative examples of mitotic inhibitors include paclitaxel, vinblastine, and docetaxel. Illustrative examples of hormonal receptor targeting agents include tamoxifen, letrozole, and flutamide. Illustrative examples of monoclonal antibodies include rituximab, trastuzumab, and bevacizumab. Illustrative immunomodulators include interferon-alpha, interleukin-2, and thalidomide. Illustrative examples of proteasome inhibitors include bortezomib and carfilzomib. Illustrative examples of kinase inhibitors include imatinib, sorafenib, and erlotinib. Illustrative examples of DNA repair inhibitors include niraparib and veliparib.

In some embodiments, at least one of the molecules in the combination therapy is administered using the substantially same dosage regimen (dose, frequency and duration of treatment) that is generally employed when the agent is used as monotherapy for treating the same cancer. Alternatively, the subject receives a lower total amount of the molecule and/or the population of hypoxia-resistant immune cells subject of the invention in the combination therapy when compared to embodiments wherein the population of hypoxia-resistant immune cells is used the sole treatment (i.e. monotherapy with the population of hypoxia-resistant immune cells). The lower total amount may be achieved by, for example, using smaller doses, less frequent doses, a shorter treatment duration, or any combination thereof.

In embodiments wherein the hypoxia-resistant immune cells described herein are administered as part of a combination therapy, said cells and the further additional active ingredient (i.e. molecule) may be administered either alone (i.e. distinct pharmaceutical compositions) or in a single medicament (i.e. a single pharmaceutical composition). In embodiments wherein a plurality of pharmaceutical compositions are used, the pharmaceutical compositions may be administered simultaneously, concurrently or sequentially in any order.

Sequential administration refers to administration of one or more dosages of the pharmaceutical composition followed after a given time interval by separate administration of another pharmaceutical composition. Sequential administration is particularly useful when the pharmaceutical compositions in the combination therapy are in different dosage forms (solid/liquid) and/or are administered on different dosing schedules, e.g. one is administered at least daily and a further one is administered less frequently, such as once weekly, once every two weeks, or once every three weeks. The time interval may be any time interval, including hours, days, weeks or months. In some embodiments sequential administration refers to administrations separated by a time interval of one of at least 10 min, 30 min, 1 hour, 6 hours, 8 hours, 12 hours, 24 hours, 36 hours, 48 hours, 3 days, 4 days, 5 days, 6 days, 1 week, 2 weeks, 3 weeks, 1 month, 6 weeks, 2 months, 3 months, 4 months, 5 months or 6 months.

A further aspect of the invention is directed to kit of parts that comprise means to generate a population of hypoxia-resistant immune cells wherein Drp1 expression and/or activity is directly reduced or inhibited and wherein the kit of parts further comprises instructions for use, and optionally means for storing said cells. The envisaged kit typically includes one or more containers, preferably a plurality of containers, packaging material, and a label or package insert on or associated with the container or containers and/or packaging, generally including instructions for generation of the hypoxia-resistant immune cells. It is understood that such kit of parts at least comprise a Drp1 inhibitor as described herein, or a combination of distinct Drp1 inhibitors.

The terms “kit of parts” and “kit” as used herein refer to a product containing components necessary for carrying out the methods (e.g. production of a population of hypoxia-resistant immune cells and/or administration of said cells to a subject), packed so as to allow their transport and storage. Materials suitable for packing the components comprised in a kit include crystal, plastic (e.g. polyethylene, polypropylene, polycarbonate), bottles, flasks, vials, ampules, paper, envelopes, or other types of containers, carriers or supports. Where a kit comprises a plurality of components, at least a subset of the components (e.g. two or more of the plurality of components) or all of the components may be physically separated, e.g. comprised in or on separate containers, carriers or supports.

The components comprised in a kit may be sufficient or may not be sufficient for carrying out the specific methods described herein, such that external reagents or substances may not be necessary or may be necessary for performing the methods, respectively. Typically, kits are employed in conjunction with standard laboratory equipment, such as liquid handling equipment, environment (e.g. temperature) controlling equipment, analytical instruments, etc. In addition to the recited set of components as taught, the present kits may also include some or all of solvents, buffers. Examples of solvents of buffers include without limitation histidine-buffers, citrate-buffers, succinate-buffers, acetate-buffers, phosphate-buffers, formate buffers, benzoate buffers, TRIS (Tris(hydroxymethyl)-aminomethan) buffers or maleate buffers, or mixtures thereof. Additionally or alternatively, the kit of parts may include enzymes, detectable labels, detection reagents, and control formulations (positive and/or negative), useful in the method subject of the invention. The terms may be used interchangeably with the term “article of manufacture”, which broadly encompasses any man-made tangible structural product, when used in the present context. Typically, the kits may also include instructions for use thereof, such as on a printed insert or on a computer readable medium. The kit may further comprise documents regarding safety, documents concerning quality assurance and any other information that is commonly provided in kit of parts.

Further provided are kit of parts for the administration of the hypoxia-resistant immune cells wherein Drp1 expression and/or activity is directly reduced or inhibited to subjects in need thereof, and optionally means for storing said cells. The envisaged kit typically includes one or more containers, preferably a plurality of containers, packaging material, and a label or package insert on or associated with the container or containers and/or packaging, generally including instructions for administration of the cells to a subject. In embodiments wherein a plurality of containers is present in the kit, preferably each container contains a single unit dose of the hypoxia-resistant immune cells. The unit dose may be an amount or number of the cells to be administered to the subject in a first dose or a multiplication of the number (or more) the cells to be administered in multiple consecutive doses. Alternatively, the container may comprise the minimal effective number of cells that would be administered to the subject in connection with the administration method. In some embodiments, the unit dose is the minimum number of cells or number of cells that would be administered in a single dose to any subject in need thereof. For example, the unit dose in some aspects may include a minimum number of cells that would be administered to a subject having a relatively low disease burden. In some embodiments, the cells have been derived from the subject to be treated by methods as provided herein or in need thereof. In some of the same and other embodiments, the number of cells in the unit dose is the number of cells or number of recombinant receptor-expressing or CAR-expressing cells that is considered an optimal dose to administer to a particular subject in a first dose, such as a subject from which the cells have been derived. In some embodiments, the cells have been derived from the subject prior to treatment by methods as provided herein.

Optionally and in embodiments wherein the kit comprises a plurality of containers comprising the hypoxia-resistant immune cells, each of the containers comprise or substantially comprise the same or substantially the same number of cells.

Suitable containers comprised in the kit include without limitation bottles, vials, syringes, and flexible bags, such as infusion bags. In particular embodiments, the containers are bags, e.g., flexible bags, such as those suitable for infusion of cells to subjects, e.g., flexible plastic or PVC bags, and/or IV solution bags. The bags in some embodiments are sealable and/or able to be sterilized, so as to provide sterile solution and delivery of the cells and compositions. In some embodiments, the containers, e.g., bags, have a capacity of at or about or at least at or about 10, 20, 30, 40, 50, 60, 70, 80, 90, 100, 200, 300, 400, 500, or 1000 ml capacity, such as between at or about 10 and at or about 100 or between at or about 10 and at or about 500 mL capacity. In some embodiments, the containers, e.g., bags, are and/or are made from material which is stable and/or provide stable storage and/or maintenance of cells at one or more of various temperatures, such as in cold temperatures, e.g. below at or about or at or about −20° C., −80° C., −120° C., 135° C. and/or temperatures suitable for cryopreservation, and/or other temperatures, such as temperatures suitable for thawing the cells and body temperature such as at or about 37° C., for example, to permit thawing, e.g., at the subjects location or location of treatment, e.g. at bedside, immediately prior to treatment. The containers described herein may be manufactured from a variety of materials such as glass or plastic. In some embodiments, the container has one or more port such as a sterile access port for connection of tubing or cannulation to one or more tubes, e.g., for intravenous or other infusion and/or for connection for purposes of transfer to and from other containers, such as cell culture and/or storage bags or other containers. Exemplary containers include infusion bags, intravenous solution bags, vials, including bags and vials equipped with stoppers that are pierceable by a needle for injection.

As described briefly above, the kit of parts may further include a package insert or label with one or more pieces of identifying information and/or instructions for use. In some embodiments, the information or instructions indicates that the contents can or should be used to treat a particular condition or disease, and/or providing instructions to do so. In some embodiments, the label or package insert provides instructions to treat a subject, optionally the subject from which the cells have been derived according to any of the embodiments of the provided methods. In some embodiments, the instructions specify administration, in a first dose, of one unit dose, e.g., the contents of a single individual container in the article of manufacture, followed by one or more consecutive doses at a specified time point or within a specified time window and/or after the detection of the presence or absence or amount or degree of one or more factors or outcomes in the subject.

In some embodiments, the label or package insert or packaging comprises an identifier to indicate the specific identity of the subject from which the cells are derived and/or are to be administered. In the case of autologous transfer, the identity of the subject from which the cells are derived is the same as the identity of the subject to which the cells are to be administered. Thus, the identifying information may specify that the cells are to be administered to a particular patient, such as the one from which the cells were originally derived. Such information may be present in the packaging material and/or label in the form of a bar code or other coded identifier, or may contain an indication of the name and/or other identifying characteristics of the subject.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples.

### EXAMPLES

### Example 1. Generation and Evaluation of Hypoxia-Resistant Immune Cells Having Reduced Drp1 Expression

### Material and Methods

Cell culture conditions: NK-92 cells are cultured in IL-2 supplemented, complete alpha MEM medium (12.5% foetal bovine serum, 12.5% horse serum, 2 mM L-glutamine and 1% penicillin-streptomycin). The K562 cell line is cultured in compete RPMI medium (10% foetal bovine serum, 2 mM L-glutamine and 1% penicillin-streptomycin). To stably maintain 1% O₂ while keeping a temperature of 37° C., the experiments are performed in state-of-the-art H45 HEPA Hypoxystations (Don Whitley Scientific). NK-92 cells cultured in 21% O₂ are used as baseline control samples and NK-92 cells cultured in 1% O₂ without any metabolic rewiring serve as hypoxic controls. NK cells were cultured for 48 h or 60 h in hypoxia/normoxia.

Mdivi-1 treatment: As the NK-92 cell line is expanded in vitro for scientific research applications and before infusion into the patient, this approach provides a therapeutic window to rewire the NK-92 metabolism. 10 μM of the Drp1 inhibitor ‘Mitochondrial Division Inhibitor 1 (Mdivi-1) is added directly into the medium of NK-92 cells, 72 h prior to the co-culture assays.

CRISPR editing: To introduce genetic adaptations in the genome of NK-92 cells, we use the Lonza 4D-Nucleofector System to transfer pre-assembled CRISPR-Cas9 Ribonucleoprotein complexes into the cells. sgRNA sequences were chosen based on minimal off-target editing sites. Our preliminary data indicate that a multi-guide approach, using three different sgRNA sequences in the RNP complex for each gene of interest, rendered superior KO scores.

sgRNA Sequences (Obtained from Synthego):

1)
(SEQ ID NO: 1)
AUAUUCUGUUUUCAGAGCAG
2)
(SEQ ID NO: 2)
UUCCAGUACCUCUGGGAAGC
3)
(SEQ ID NO: 3)
GAAGAUAAACGGAAAACAAC

Gene editing was validated and quantified on protein (flow cytometry) and DNA (Sanger sequencing) level. Sanger sequencing was performed at the VIB genomic service facility at uAntwerp and analysed using the Inference of CRISPR Edits (ICE) software tool (Synthego). For DRP1, KO scores of 100% were obtained.

Co-culture assays: The cytotoxic potential of control, Mdivi-1 treated, and DRP1-KO NK-92 cells was analysed after 48 h of hypoxic culturing and compared to baseline NK-92 killing with the K562 target cell line, the golden standard for NK cell-mediated killing. The above-mentioned NK-92 cells and K562 cells were cultured together for 4 or 16 hours (overnight). A flow cytometric killing assay assessing Annexin-V and 7-AAD in the PKH67-prelabeled K562 cells was used to quantify specific NK-92 cell-mediated killing. The viability of the NK-92 cells was also determined by Annexin-V and 7-AAD staining.

### Results

The protein responsible for mitochondrial fragmentation in NK cells in hypoxia, Dynamin related protein 1 (Drp-1), is overly activated in tumour-infiltrating NK cells. The resulting mitochondrial fragmentation compromises the function of NK cells in the tumour microenvironment. To address this issue, we have investigated the use of a specific inhibitor (Mdivi-1) and CRISPR-Cas9 editing to block the function of Drp-1, thereby stopping the unwanted fragmentation. Indeed, after 48 hours of hypoxic culturing, the viability of the NK-92 cells was significantly lower compared to NK-92 cells cultured in normoxia. However, the Drp-1KO cells showed a significantly higher viability after 48 hours in hypoxia compared to the unedited cells (FIG. 1 and Table 1).

TABLE 1
Viability after 48 h culturing in hypoxic
conditions. 5 repeats for each condition.

| Viability | Condition |
|---|---|
| 70% | Normoxia |
| 74% | Normoxia |
| 48% | Normoxia |
| 70% | Normoxia |
| 77% | Normoxia |
| 18% | Hypoxia |
| 7% | Hypoxia |
| 10% | Hypoxia |
| 25% | Hypoxia |
| 27% | Hypoxia |
| 12% | Mdivi-1 |
| 13% | Mdivi-1 |
| 21% | Mdivi-1 |
| 32% | Mdivi-1 |
| 35% | Mdivi-1 |
| 28% | DRP1 KO |
| 56% | DRP1 KO |
| 34% | DRP1 KO |
| 36% | DRP1 KO |

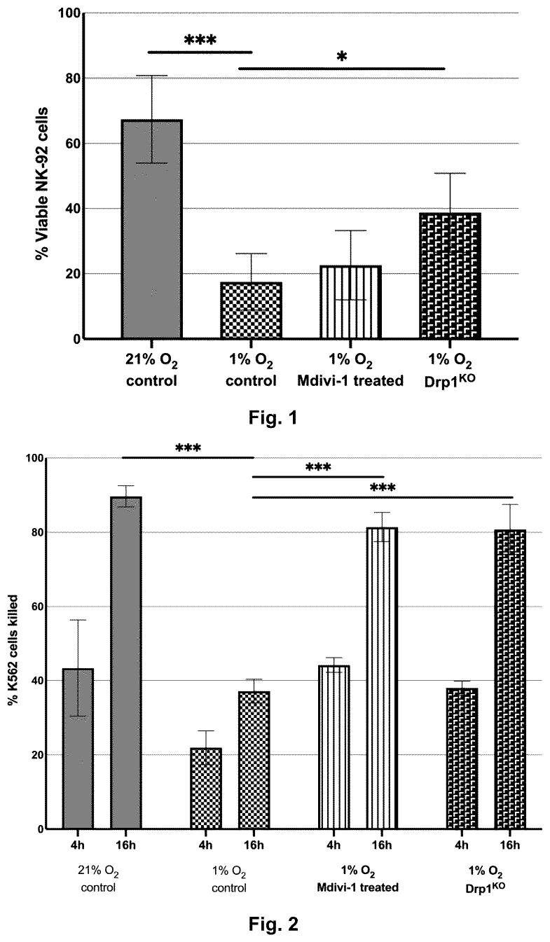

To test the effectiveness of our metabolically rewired NK cellular therapy product in hypoxic conditions, we performed a coculture experiment for 4 hours and overnight (16 hours), comparing the killing potential of normoxic NK cells to that of hypoxic NK cells (FIG. 2 and Table 2). With the overnight co-culture, we observed a significant difference between normoxic NK cells and hypoxic NK cells. Importantly, we also observed a significant difference between control hypoxic NK cells and NK cells that were either treated with Mdivi-1 or had the DRP1 gene knocked out. These results suggest that our metabolic rewiring strategy enhances the viability and killing potential of NK cells in hypoxia. This finding has important implications for the development of more effective cancer therapies. By improving the fitness of NK cells in the harsh and suppressive TME, we may be able to synergize with other forms of immunotherapy, such as CAR engineering, to increase treatment efficacy.

TABLE 2
Killing potential of normoxic NK cells and hypoxic
NK cells. 5 repeats for each condition.

| Killing | Condition | Timepoint |
|---|---|---|
| 39% | Normoxia | 4 h |
| 18% | Normoxia | 4 h |
| 14% | Normoxia | 4 h |
| 70% | Normoxia | 4 h |
| 77% | Normoxia | 4 h |
| 16% | Hypoxia | 4 h |
| 9% | Hypoxia | 4 h |
| 21% | Hypoxia | 4 h |
| 30% | Hypoxia | 4 h |
| 34% | Hypoxia | 4 h |
| 38% | Mdivi-1 | 4 h |
| 46% | Mdivi-1 | 4 h |
| 46% | Mdivi-1 | 4 h |
| 40% | Mdivi-1 | 4 h |
| 44% | Mdivi-1 | 4 h |
| 51% | Mdivi-1 | 4 h |
| 35% | Drp1 KO | 4 h |
| 38% | Drp1 KO | 4 h |
| 35% | Drp1 KO | 4 h |
| 43% | Drp1 KO | 4 h |
| 89% | Normoxia | 16 h |
| 96% | Normoxia | 16 h |
| 85% | Normoxia | 16 h |
| 78% | Normoxia | 16 h |
| 94% | Normoxia | 16 h |
| 96% | Normoxia | 16 h |
| 41% | Hypoxia | 16 h |
| 37% | Hypoxia | 16 h |
| 38% | Hypoxia | 16 h |
| 43% | Hypoxia | 16 h |
| 42% | Hypoxia | 16 h |
| 22% | Hypoxia | 16 h |
| 84% | Mdivi-1 | 16 h |
| 90% | Mdivi-1 | 16 h |
| 80% | Mdivi-1 | 16 h |
| 71% | Mdivi-1 | 16 h |
| 93% | Drp1 KO | 16 h |
| 90% | Drp1 KO | 16 h |
| 77% | Drp1 KO | 16 h |
| 63% | Drp1 KO | 16 h |

### Example 2. Cytotoxic Potential of Drp1KO NK-92 Cells

### Materials and Methods

CAR electroporation: NK-92 or Drp1^{KO} NK-92 cells were electroporated at a concentration of 4×10⁶ cells/mL in the presence of 100 g/mL CD70-CAR-IL-15-encoding mRNA using the Lonza 4D nucleofector device. NK-92 cells electroporated without CAR-encoding mRNA (MOCK) were used as control cells.

4 h co-culture assays: Cytotoxic activity towards the Raji CD70⁺ tumor cell line was assessed by co-culturing (CAR) NK cells 48 h after CAR mRNA electroporation and hypoxic/normoxic culturing with PKH67-labeled CD70⁺ Raji cells in a 5:1 effector:target ratio. After 4 h, co-cultures were stained with 7-AAD and Annexin V-PE. Target cell survival was measured on a CytoFLEX flow cytometer. 24 h longitudinal cytotoxicity measurements: The longitudinal cytotoxic activity of (CAR) NK cells towards the CD70⁺ solid tumour cell lines Panc-1 (pancreatic ductal adenocarcinoma), HeLa (cervical squamous carcinoma), LIM2099 (colorectal adenocarcinoma) and SC263 (head and neck squamous carcinoma) was analyzed using the xCELLigence Real-Time Cell Analysis (RTCA; Agilent) that records cell viability and growth by impedance measurements. Tumour cells were seeded in gold-coated 16-well plates overnight and treated with (CAR) NK cells in a 1:1 and 1:5 effector:target ratio. CAR NK cells were electroporated with CAR mRNA and cultured in normoxia/hypoxia 48 h before co-culture. The impedance was monitored by automated measurements, expressed as Cell Index (CI) and normalized to 1 after starting the co-culture.

### Results

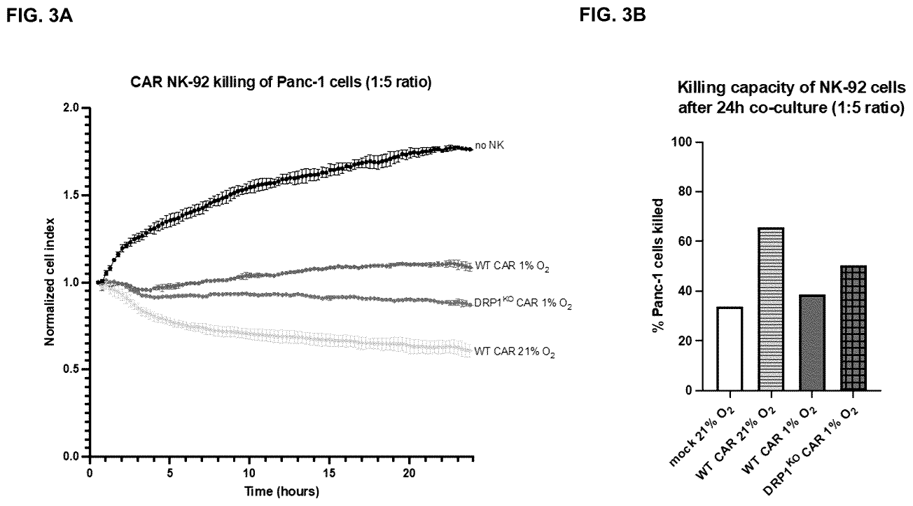

It could be observed that Drp1^{KO} CAR NK-92 cells have a better Panc-1 cell killing capacity after hypoxic culturing compared to control NK-92 cells in a 1:5 ratio experiment (FIG. 3). The killing capacity observed in the different conditions are summarized in Table 3.

TABLE 3
Endpoint xCELLigence Panc-1 killing experiments 1:5 tumour:NK
cell ratio. Numbers depict % killing of tumor cells.

| | Mock | WT CAR 21% O₂ | WT CAR 1% O₂ | DRP1^{KO} 1% O₂ |
|---|---|---|---|---|
| Panc-1 | 33.73 | 65.63 | 38.62 | 50.63 |

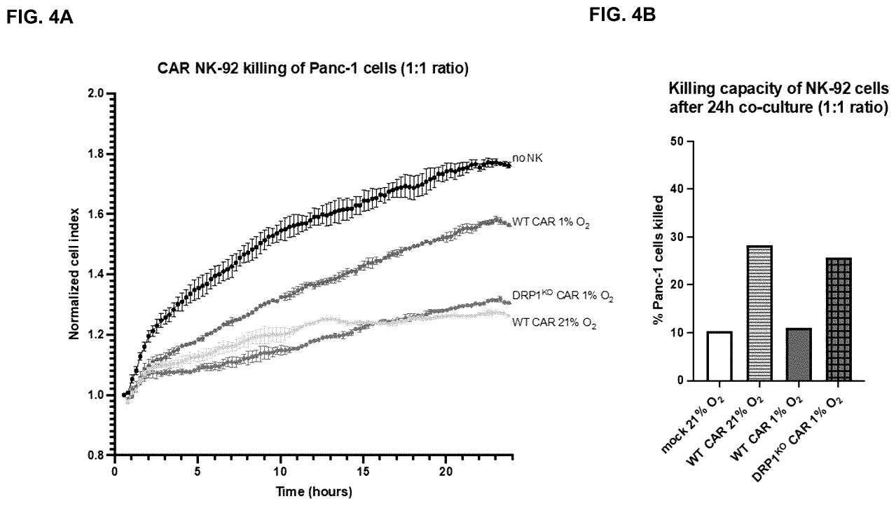

In addition, it could be observed that Drp1^{KO} CAR NK-92 cells have a better Panc-1 cell killing capacity after hypoxic culturing compared to control NK-92 cells in a 1:1 ratio experiment (FIG. 4). The killing capacity observed in the different conditions are summarized in Table 4.

TABLE 4
Endpoint xCELLigence Panc-1 killing experiments 1:1 tumour:NK
cell ratio. Numbers depict % killing of tumor cells.

| | Mock | WT CAR 21% O₂ | WT CAR 1% O₂ | DRP1^{KO} 1% O₂ |
|---|---|---|---|---|
| Panc-1 | 10.40 | 28.23 | 11.00 | 25.67 |

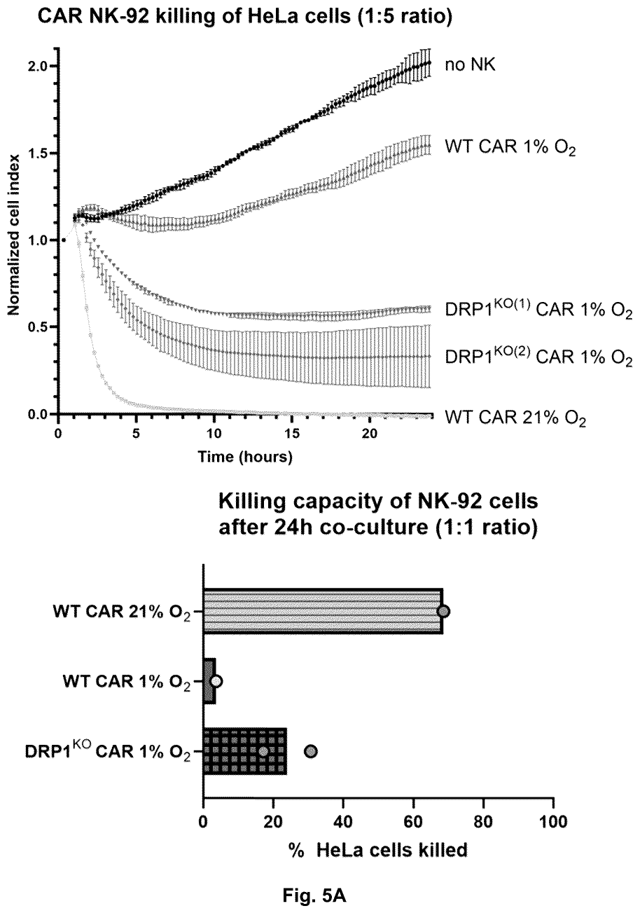

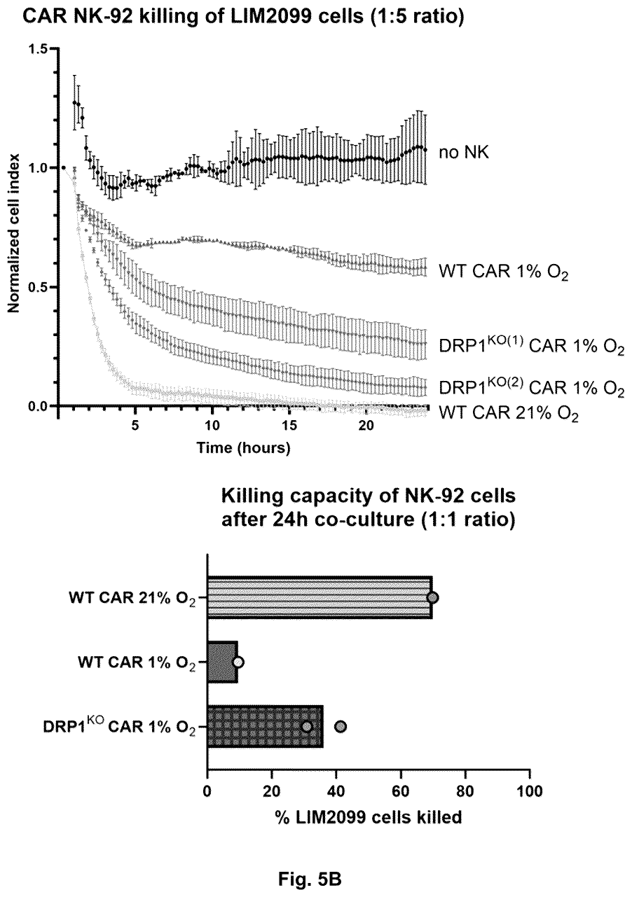

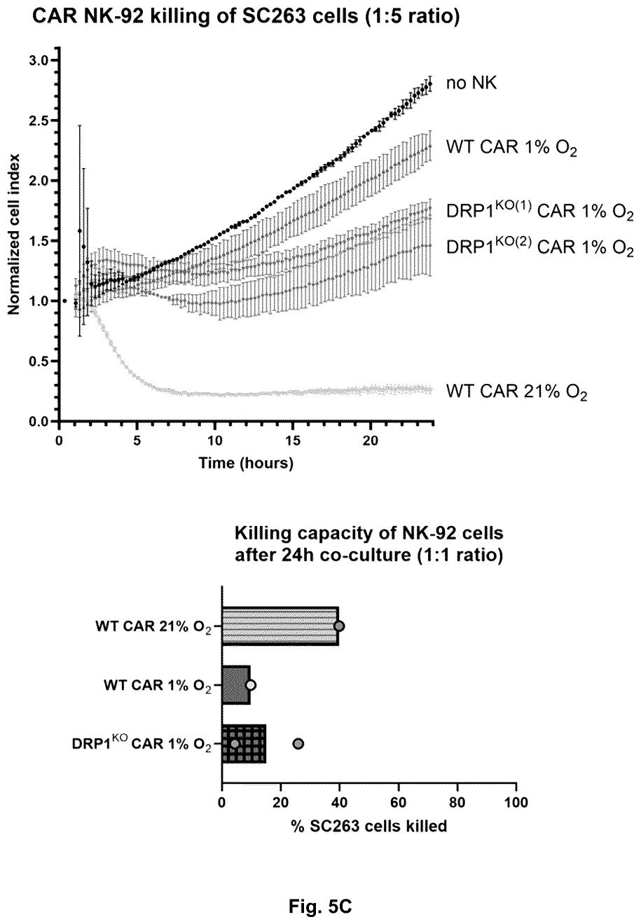

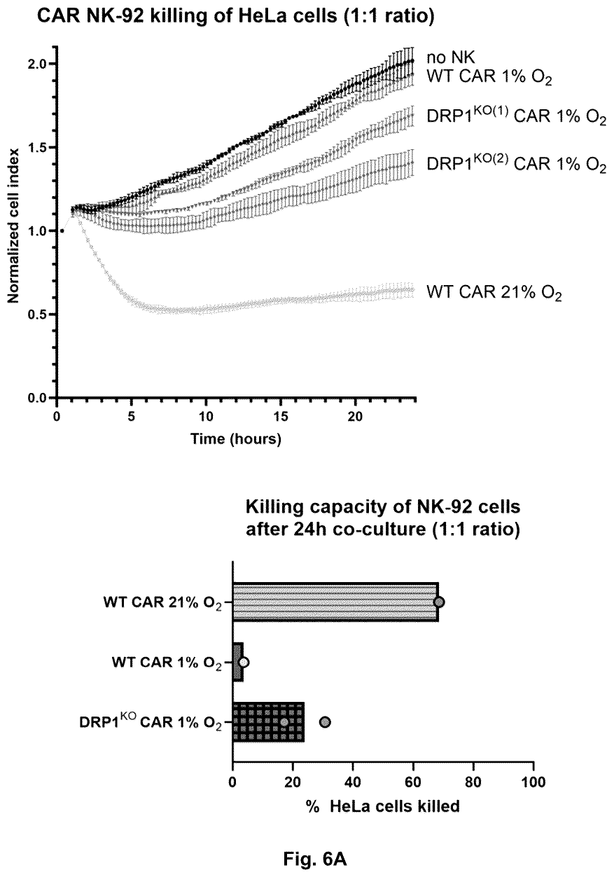

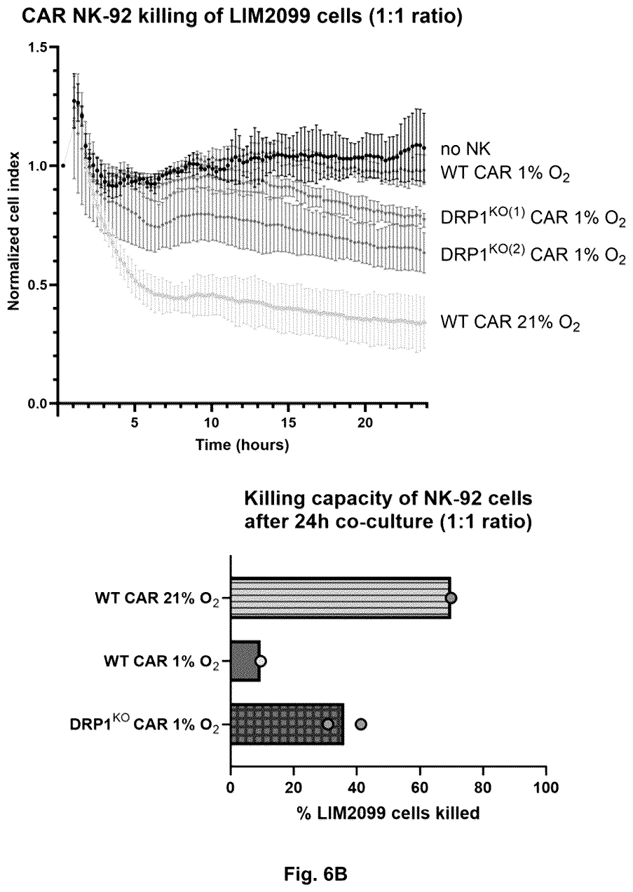

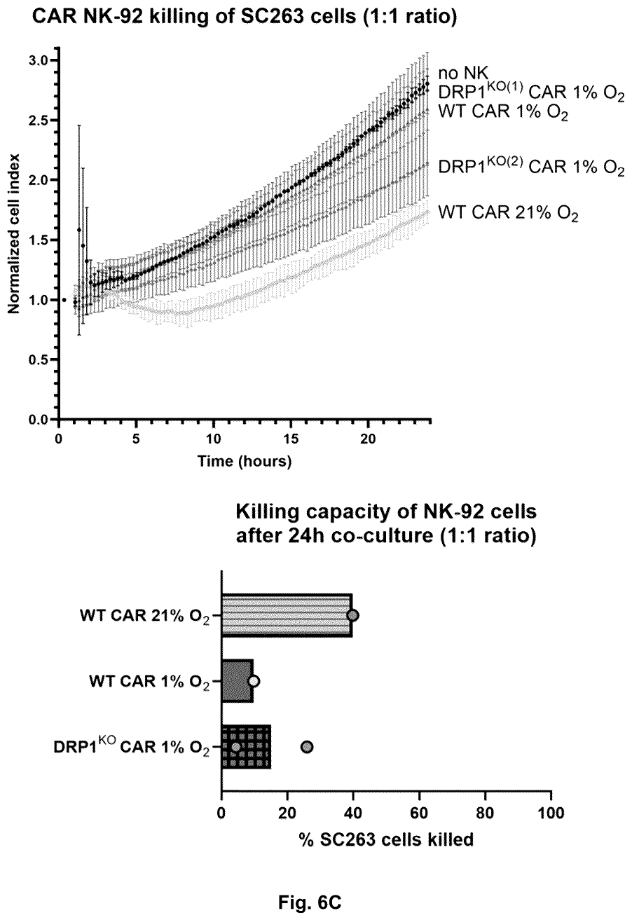

These results were replicated in other solid tumour cell lines HeLA, LIM2099 and SC263 (FIGS. 5 and 6). The killing capacity observed in these conditions are summarized in Tables 5 and 6.

TABLE 5
Endpoint xCELLigence solid tumour killing experiments 1:5 tumour:NK
cell ratio. Numbers depict % killing of tumor cells.

| | WT CAR 21% O₂ | WT CAR 1% O₂ | DRP1^{KO} 1% O₂ |
|---|---|---|---|
| HeLa | 100.00 | 10.60 | 73.73 |
| LIM2099 | 100.00 | 47.22 | 85.61 |
| SC263 | 91.53 | d21.42 | 44.95 |

TABLE 6
Endpoint xCELLigence solid tumour killing experiments 1:1 tumour:NK
cell ratio. Numbers depict % killing of tumor cells.

| | WT CAR 21% O₂ | WT CAR 1% O₂ | DRP1^{KO} 1% O₂ |
|---|---|---|---|
| HeLa | 68.57 | 3.65 | 23.89 |
| LIM2099 | 69.87 | 9.53 | 36.07 |
| SC263 | 39.80 | 9.76 | 15.15 |

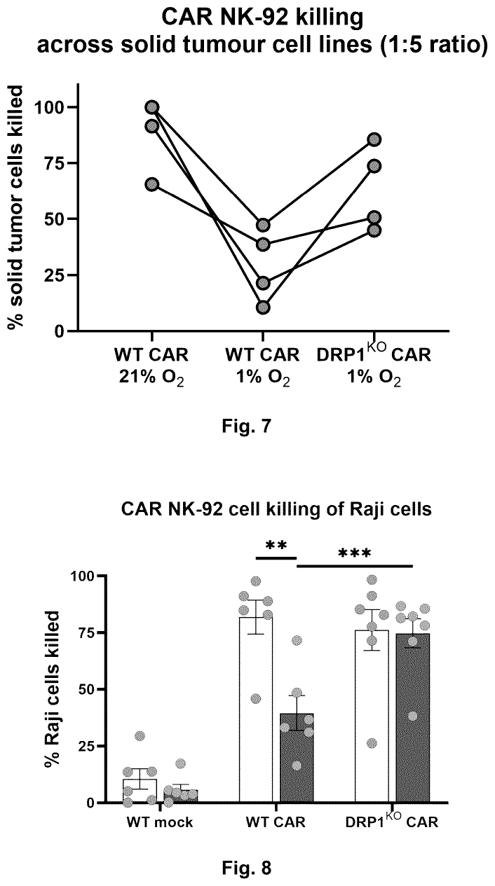

Altogether, the experiments across different solid tumour cell lines demonstrate that Drp1^{KO} CAR NK-92 withstand hypoxic deterioration, leading to a better killing of solid tumour cells (FIG. 7).

Finally, Drp1^{KO} CAR NK-92 cells are characterized by a better Raji cell killing capacity after hypoxic culturing compared to control NK-92 cells (FIG. 8). The killing capacity observed in the different conditions are summarized in Table 7.

TABLE 7
CAR NK-92 mediated killing of Raji cells in a co-
culture experiment. Left and right column in each
O₂ condition represent biological replicates.

| Killing of Raji cells (%) | |
|---|---|
| 21% O₂ | 1% O₂ |

| | | | | |
|---|---|---|---|---|
| WT mock | 13.53 | 1.17 | 3.20 | 0.17 |
| WT CAR | 97.72 | 91.15 | 71.59 | 31.00 |
| DRP1^{KO} CAR | 98.34 | 91.25 | 78.23 | 82.17 |

1-15. (canceled)

16. A method of treating cancer in a subject, the method comprising:
administering to the subject a therapeutically effective amount of a population of redirected immune cells modified so as to have a 50% decrease in Drp1 expression and/or activity as compared to otherwise identical immune cells not comprising the modification of Drp1 expression and/or activity.

17. The method according to claim 16, wherein the redirected immune cells are administered as a pharmaceutical composition.

18. The method according to claim 16, wherein the cancer is a solid tumour or haematological malignancy.

19. The method according to claim 16, wherein the redirected immune cells are chimeric antigen receptor (CAR) redirected immune cells, monospecific affinity ligand redirected immune cells, or multispecific affinity ligand redirected immune cells.

20. The method according to claim 19, wherein the multispecific affinity ligand redirected immune cells are bispecific antibody redirected immune cells.

21. The method according to claim 16, wherein the Drp1 expression and/or activity is directly reduced or inhibited by genomic modification of the DNM1L gene or DNM1L promoter sequence.

22. The method according to claim 21, wherein the genomic modification of the DNM1L gene or DNM1L promoter sequence results in a complete and permanent ablation of Drp1 expression.

23. The method according to claim 16, wherein the redirected immune cells are human redirected immune cells and the subject is a human subject.

24. The method according to claim 16, wherein the redirected immune cells are natural killer (NK) cells or T cells.

25. The method according to claim 16, wherein the redirected immune cells are further modified for survival and/or efficacy in a solid tumour microenvironment.

26. The method according to claim 16, wherein Drp1 expression and/or activity is reduced by at least 50%, as compared to immune cells that, in absence of any modification of Drp1 expression and/or activity, share a genomic, transcriptomic, and/or proteomic identity with the redirected immune cells.

27. The method according to claim 16, further comprising producing redirected immune cells modified so as to have at least a 50% decreased Drp1 expression and/or activity as compared to an otherwise identical cell not comprising the modification of Drp1 expression and/or activity, the method further comprising:
(a) providing redirected immune cells;
(b) treating the redirected immune cells ex vivo with a specific Drp1 inhibitor and
(c) administering the treated redirected immune cells to the subject.

28. The method according to claim 27, wherein the specific Drp1 inhibitor is an inhibitor selected from the group consisting of: a chemical inhibitor, a binding protein, a gene editing system, and an antisense agent.

29. The method according to claim 28, wherein the Drp1 expression and/or activity is reduced by at least 75%, by the specific Drp1 inhibitor.

30. A method of producing a redirected immune cell modified so as to have an at least 50% decreased Drp1 expression and/or activity as compared to an otherwise identical cell not comprising the modification of Drp1 expression and/or activity, the method comprising:
providing redirected immune cells;
treating the redirected immune cells ex vivo with a specific Drp1 inhibitor, so as to obtain modified and redirected immune cells having at least 50% decreased Drp1 expression and/or activity compared to the cells prior to the treatment.

31. The method according to claim 30, wherein the specific Drp1 inhibitor is an inhibitor selected from the group consisting of: a chemical inhibitor, a binding protein, a gene editing system, and an antisense agent.

32. The method according to claim 30, wherein the Drp1 expression and/or activity is reduced by at least 50% by the specific Drp1 inhibitor.

33. The method according to claim 30, wherein the Drp1 expression and/or activity is reduced by at least 75%, by the specific Drp1 inhibitor.

34. The method according to claim 30, wherein the redirected immune cells are natural killer (NK) cells.
